(19) United States
(12) Patent Application Publication
(10) Pub. No.: US 2026/0354442 A1
(43) Pub. Date: Nov. 12, 2026

(54) RESINATE COMPLEXES FOR USE IN ORAL PRODUCTS

(71) Applicant: RAI Strategic Holdings, Inc., Winston-Salem, NC (US)

(72) Inventors: Michael Andrew Zawadzki, Clemmons, NC (US); Ryan Mills, Greensboro, NC (US)

(21) Appl. No.: 19/666,428

(22) Filed: May 4, 2026

Related U.S. Application Data
(60) Provisional application No. 63/799,849, filed on May 5, 2025.

(51) Int. Cl. A24B 15/38; A24B 15/24; A24B 15/30; A24B 15/40

(52) U.S. Cl. CPC A24B 15/385; A24B 15/243; A24B 15/303; A24B 15/308; A24B 15/403

(57) ABSTRACT

Resinate complexes and products for oral use including a resinate complex are described herein. The resinate complex includes a resin base component and one or more active agents associated therewith. Various types of oral products, such as pastilles, lozenges, tablets, gums, and pouched products are described. Methods for preparing such resinate complexes and products are also provided herein.

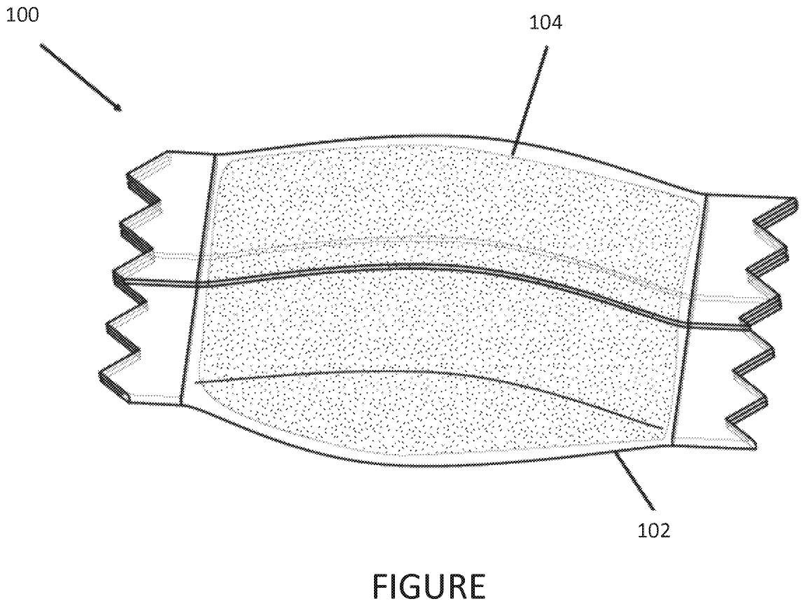

### CROSS-REFERENCE TO RELATED APPLICATIONS

The present application claims priority to U.S. Provisional Patent Application No. 63/799,849, filed May 5, 2025, the disclosure of which is incorporated herein by reference in its entirety

### FIELD OF THE DISCLOSURE

The present disclosure relates to components for use in oral compositions, corresponding oral compositions and products, and methods for forming the components, oral compositions, and products.

### BACKGROUND

There are many categories of products intended for oral use and enjoyment. For example, oral tobacco products containing nicotine, which is known to have both stimulant and anxiolytic properties, have been available for many years. Conventional formats for so-called “smokeless” tobacco products include moist snuff, snus, and chewing tobacco, which are typically formed almost entirely of particulate, granular, or shredded tobacco, and which are either portioned by the user or presented to the user in individual portions, such as in single-use pouches or sachets. See for example, the types of smokeless tobacco formulations, ingredients, and processing methodologies set forth in U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and 7,694,686 to Atchley et al.; U.S. Pat. No. 7,810,507 to Dube et al.; U.S. Pat. No. 7,819,124 to Strickland et al.; U.S. Pat. No. 7,861,728 to Holton, Jr. et al.; 7,901,512 to Quinter et al.; U.S. Pat. No. 8,627,828 to Strickland et al.; U.S. Pat. No. 11,246,334 to Atchley, each of which is incorporated herein by reference. In addition, traditional tobacco materials and non-tobacco materials have been combined with other ingredients to form product formats distinct from traditional smokeless products, with example formats including lozenges, pastilles, gels, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

There is continuing interest in the development of new types of oral products that deliver advantageous sensorial or biological activity. Such products typically contain flavorants and/or active agents such as nicotine, caffeine, botanicals, or cannabidiol. The format of such products can vary and include pouched products containing a powdered or granular composition, lozenges, pastilles, liquids, gels, emulsions, meltable compositions, and the like. See, for example, the types of products described in US Patent App. Pub. Nos. 2022/0160675 to Gerardi et al.; 2022/0071984 to Poole et al.; 2021/0378948 to Gerardi et al.; 2021/0330590 to Hutchens et al.; 2021/0186081 to Gerardi et al.; 2021/0177754 to Keller et al; 2021/0177043 to Gerardi et al.; 2021/0177038 to Gerardi et al.; 2021/0169867 to Holton, Jr. et al.; 2021/0169792 to Holton, Jr. et al.; 2021/0169132 to Holton, Jr. et al.; 2021/0169121 to St. Charles, and 2021/0169122 to St. Charles, each of which is incorporated herein by reference.

### BRIEF SUMMARY

The present disclosure is generally directed to resinate complexes and oral compositions and products comprising such resinate complexes. The resinate complexes generally comprise a resin base component and an active agent associated with the resin base component. In some embodiments, the active agent comprises at least a substituted 3-(1-methylpyrrolidin-2-yl)pyridine.

The present disclosure includes, without limitation, the following embodiments.

Embodiment 1: An oral product comprising a resinate complex, the resinate complex comprising a resin base component and one or more active agents associated with the resin base component, selected from:
(i) a substituted 3-(1-methylpyrrolidin-2-yl)pyridine having a structure according to Formula I:

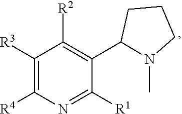

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen;
(ii) a substituted 3-(1-methylpyrrolidin-2-yl)pyridine having a structure according to Formula II:

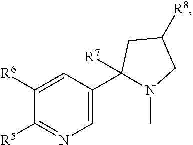

wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; R⁷ is selected from the group consisting of hydrogen and CH₃; R⁸ is selected from the group consisting of hydrogen and C₁-C₃ alkyl; and at least one of R⁷ and R⁸ is not hydrogen; and
(iii) a 3-(azetidin-2-yl)pyridine or 3-(azetidin-2-ylmethoxy)pyridine having a structure according to Formula III:

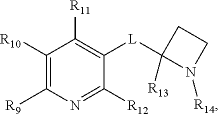

wherein L is a bond or —OCH₂—*, where the asterisk indicates an attachment point to the azetidine ring; R⁹, R¹⁰, R¹¹, and R¹² are each independently selected from the group consisting of hydrogen, alkyl, alkoxy, halogen, and cyano; R¹³ is H or CH₃; and R¹⁴ is H or CH₃.

Embodiment 2: The oral product of Embodiment 1, wherein the active ingredient has a structure according to Formula I, optionally wherein R¹, R², and R³ are each H, and R⁴ is optionally substituted C₁-C₆ alkyl, F, Cl, Br, OCH₃, OEt, or CN, or optionally wherein R¹, R², and R³ are each H, and R⁴ is C₁-C₃ alkyl.

Embodiment 3: The oral product of Embodiment 1, wherein the active ingredient has a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

Embodiment 4: The oral product of Embodiment 1, wherein the active ingredient has a structure according to Formula III, optionally wherein R⁹ is H or CH₃; and R¹⁰, R¹¹, and R¹² are each H.

Embodiment 5: The oral product of any one of Embodiments 1-4, wherein the oral product further comprises a flavorant (e.g., a sensate), a salt, a colorant, a sweetener, a pH-adjusting component (buffering agent or pH adjuster), or any combination thereof.

Embodiment 6: The oral product of any one of Embodiments 1-5, wherein the oral product further comprises a filler, binder, disintegration aid, antioxidant, humectant, preservative, or any combination thereof.

Embodiment 7: The oral product of any of Embodiments 1-6, wherein the oral product further comprises one or more additional active agents selected from the group consisting of a nicotine component, a cannabinoid, a terpene, caffeine, an amino acid, a vitamin, melatonin, a botanical extract, or any combination thereof.

Embodiment 8: The oral product of any of Embodiments 1-7, wherein the oral product further comprises a nicotine component.

Embodiment 9: The oral product of any of Embodiments 1-7, wherein the oral product is substantially free of a nicotine component.

Embodiment 10: The oral product of any of Embodiments 1-9, in a form selected from the group consisting of a tablet, pastille, pouched product, gel, chewing gum, and meltable product.

Embodiment 11: The oral product of Embodiment 10, in the form of a dissolvable tablet.

Embodiment 12: The oral product of Embodiment 10, in the form of a pouched product, the pouched product comprising an outer, water-permeable pouch defining a cavity and an oral composition for oral use situated within the cavity, wherein the oral composition comprises the resinate complex.

Embodiment 13: The oral product of Embodiment 12, wherein the oral composition further comprises one or more fillers in an amount of about 50% or more by weight.

Embodiment 14: The oral product of any of Embodiments 1-13, wherein the oral composition further comprises a portion of the one or more active agents in released form, such that they are not associated with the resin base component.

Embodiment 15: The oral product of Embodiment 14, wherein the one or more active agents in released form are in a form selected from free base form, salt form, co-crystal form, salt co-crystal form, ion-paired form, and combinations thereof.

Embodiment 16: A resinate complex, the resinate complex comprising a resin base component and one or more active agents associated with the resin base component, selected from:
(i) a substituted 3-(1-methylpyrrolidin-2-yl)pyridine having a structure according to Formula I:

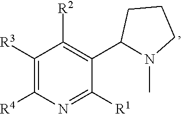

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen;
(ii) a substituted 3-(1-methylpyrrolidin-2-yl)pyridine having a structure according to Formula II:

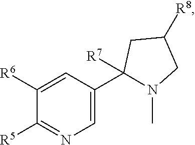

wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; R⁷ is selected from the group consisting of hydrogen and CH₃; R⁸ is selected from the group consisting of hydrogen and C₁-C₃ alkyl; and at least one of R⁷ and R⁸ is not hydrogen; and
(iii) a 3-(azetidin-2-yl)pyridine or 3-(azetidin-2-ylmethoxy)pyridine having a structure according to Formula III:

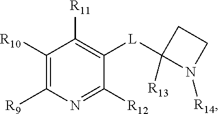

wherein L is a bond or —OCH₂—*, where the asterisk indicates an attachment point to the azetidine ring; R⁹, R¹⁰, R¹¹, and R¹² are each independently selected from the group consisting of hydrogen, alkyl, alkoxy, halogen, and cyano; R¹³ is H or CH₃; and R¹⁴ is H or CH₃.

Embodiment 17: The resinate complex of Embodiment 16, wherein the active ingredient has a structure according to Formula I, optionally wherein R¹, R², and R³ are each H, and R⁴ is optionally substituted C₁-C₆ alkyl, F, Cl, Br, OCH₃, OEt, or CN, or optionally wherein R¹, R², and R³ are each H, and R⁴ is C₁-C₃ alkyl.

Embodiment 18: The resinate complex of Embodiment 16, wherein the active ingredient has a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

Embodiment 19: The resinate complex of Embodiment 16, wherein the active ingredient has a structure according to Formula III, optionally wherein R⁹ is H or CH₃; and R¹⁰, R¹¹, and R¹² are each H.

These and other features, aspects, and advantages of the present disclosure will be apparent from a reading of the following detailed description together with the accompanying FIGURE, which is briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

### BRIEF DESCRIPTION OF THE DRAWING

Having thus described aspects of the disclosure in the foregoing general terms, reference will now be made to the accompanying drawing, which is not necessarily drawn to scale. The drawing is exemplary only and should not be construed as limiting the disclosure.

The drawing is a perspective view of a pouched product embodiment according to an example embodiment of the present disclosure, including a pouch or fleece at least partially filled with a composition configured for oral use.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Reference to “dry weight percent” or “dry weight basis” refers to weight on the basis of dry ingredients (i.e., all ingredients except water). All weight percent values herein are dry weight percent unless otherwise indicated.

Unless otherwise defined herein, by “substantially free” it is meant that the noted component (e.g., nicotine, acid, or a botanical material) has not been intentionally added, beyond trace amounts that may be present e.g., as an impurity in another component, or small amounts which may be present in certain flavor packages. For example, some embodiments can have less than 0.01% by weight of the noted component, or less than 0.001%, or even 0% by weight of the noted component, based on the weight of the disclosed oral composition or product on a dry weight basis. In some embodiments, the oral composition or product provided herein is completely free of the noted component (i.e., having 0% or as having an amount below the limit of detection).

The present disclosure is generally directed to resinate complexes and to oral compositions and products that comprise a resinate complex. Resinate complexes according to the present disclosure comprise a resin base component and one or more active agents as defined herein. Oral products and compositions can optionally further comprise various other components including, but not limited to binders, fillers/carriers, flavorants, colorants, further active agents, humectants, salts, sweeteners, pH adjusters, and the like.

A resinate complex as provided herein is generally a polymeric resin having one or more types of active agent associated therewith, e.g., via covalent bonding, ionic bonding, hydrogen bonding, or any combination thereof. In some embodiments, the polymeric resin can be considered to be a delivery vehicle for the one or more active agents. In some embodiments, the inclusion of a given active agent within an oral product or composition in the form of a resinate complex as described herein can provide greater stability than inclusion of that active agent alone. The amount of active agent associated with the polymeric resin in a given resinate complex can vary and can, in some embodiments, be tailored to achieve the desired active agent content in a given oral product.

### Resin Base Component

The resin base component can vary within the general scope of the present disclosure, but is generally a polymer. In some embodiments, the polymer of a resin base component can be any polymer (including homopolymers or all types of copolymers) with acidic functionalities, e.g., a polymeric cation exchange resin. In some embodiments, the resin base component is a weak carboxylic acid resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phophonic acids, phosphoric acids, iminodiacetic acids, and phenolic groups (e.g., as disclosed in Adams et al., J. Soc. Chem. Ind. 54, IT (1935), which is incorporated herein by reference).

Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrine. In some embodiments, the polymer comprises cross-linked moieties. Various such exchange resins are known in the art, including, but not limited to, DuPont™ Amberlite™ IRP64, DuPont™ Amberlite™ IRP69, Purolite™ C115HMR, or Doshion™ P551. See, for example, U.S. Pat. No. 3,901,248 to Lichtneckert et al., which is incorporated herein by reference. In some embodiments, the resin base component is a sulfonic acid resin.

### Active Agent Associated Therewith

The active agent of the resinate complexes of the disclosure includes, but is not limited to, a substituted 3-(1-methylpyrrolidin-2-yl)pyridine; a substituted or unsubstituted 3-azetidin-2-yl)pyridine, or a substituted or unsubstituted 3-(azetidin-2-ylmethoxy)pyridine. Alternatively (or in addition), the active agent may include components such as botanical materials, cannabinoids, terpenes, vitamins, melatonin, caffeine, nicotine compounds, tobacco extracts, or combinations thereof. The various components are described further herein below.

It is noted that the disclosure is focused on oral products wherein the active agent(s) is associated with the resin base component in the form of a resinate complex; however, certain oral products in non-limiting embodiments of the disclosure may further comprise one or more of the disclosed active agents in a different form (i.e., not associated with a resin base component as described herein). As such, some embodiments can comprise a first active agent in the form of a resinate complex and a second active agent, which can be in the form of a resinate complex or in a different form, e.g., an uncomplexed form (wherein the second active agent can be the same as or different than the first active agent).

In some embodiments, the oral products of the disclosure can be characterized as completely free or substantially free of nicotine (3-(1-methylpyrrolidin-2-yl)pyridine). By “substantially free” it is meant that no nicotine has been intentionally added, beyond trace amounts that may be present e.g., as an impurity in another component, including as a minor impurity in substituted 3-(1-methylpyrrolidin-2-yl)pyridine. For example, some embodiments can be characterized as having less than 0.01% by weight of nicotine, or less than 0.001% by weight of nicotine, or less than 0.0001%, or even 0% by weight of nicotine, calculated as the free base and based on the total weight of the oral composition or product on a dry weight basis. In some embodiments, the oral composition or product of the disclosure is completely free of (R)-, (S)-, and (R S)-3-(1-methylpyrrolidin-2-yl)pyridine (e.g., having 0% by weight of nicotine, including racemic nicotine and nicotine enantiomers, calculated as the free base and based on the total weight of the oral composition or product). In some embodiments, oral compositions and products of the disclosure do not contain any compounds obtained by chemical reactions utilizing nicotine as a starting material.

### Substituted 3-(1-methylpyrrolidin-2-yl)pyridine

As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring. In some embodiments, substituted 3-(1-methylpyrrolidin-2-yl)pyridine present is not synthetically derived from nicotine.

In some embodiments, the substituted 3-(1-methylpyrrolidin-2-yl)pyridine has a structure according to Formula I:

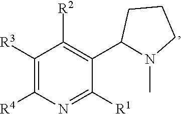

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen.

Substituted 3-(1-methylpyrrolidin-2-yl)pyridines according to Formula I with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C₁-C₆ alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is CH₃. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

Without wishing to be bound by any particular theory, it is believed that certain substitutions for hydrogen on the pyridine ring of the substituted 3-(1-methylpyrrolidin-2-yl)pyridine compound of Formula I preserve the general pharmacological profile and physiological effects of nicotine while offering the potential for one or more of greater potency, reduced product consumption, more rapid and/or complete absorption, and the like. Particularly, it is believed that in some embodiments, substituted 3-(1-methylpyrrolidin-2-yl)pyridines of the disclosure according to Formula I are readily absorbed through oral mucosa by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine of Formula I is 2-methyl-5-(1-methylpyrrolidin-2-yl)pyridine.

The substituted 3-(1-methylpyrrolidin-2-yl)pyridine (e.g., 2-methyl-5-(1-methylpyrrolidin-2-yl)pyridine) of Formula I may be present as a single enantiomer or as a mixture of enantiomers. In some embodiments, the substituted 3-(1-methylpyrrolidin-2-yl)pyridine is present in racemic form, meaning there are equal amounts of (R)- and (S)-enantiomers present. In some embodiments, the resinate complex comprises unequal amounts of (R)- and (S)-enantiomer (i.e., is enriched in either the (R)- or (S)-enantiomer. In some embodiments, the oral composition or product predominantly comprises the substituted 3-(1-methylpyrrolidin-2-yl)pyridine (e.g., 2-methyl-5-(1-methylpyrrolidin-2-yl)pyridine) in the (R)-configuration, for example, about 90% or more of the total quantity of substituted 3-(1-methylpyrrolidin-2-yl)pyridine present is in the (R)-configuration. In some embodiments, the oral composition or product predominantly comprises the substituted 3-(1-methylpyrrolidin-2-yl)pyridine (e.g., 2-methyl-5-(1-methylpyrrolidin-2-yl)pyridine) in the (S)-configuration, for example, about 90% or more of the total quantity of substituted 3-(1-methylpyrrolidin-2-yl)pyridine present is in the (S)-configuration. In some embodiments, the oral composition or product comprises 95% or more of the (S)-configuration of the substituted 3-(1-methylpyrrolidin-2-yl)pyridine (e.g., 2-methyl-5-(1-methylpyrrolidin-2-yl)pyridine), based on the total amount of substituted 3-(1-methylpyrrolidin-2-yl)pyridine present.

In some embodiments, the substituted 3-(1-methylpyrrolidin-2-yl)pyridine has a structure according to Formula II:

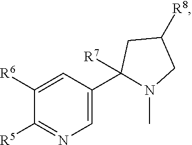

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and
   at least one of R⁷ and R⁸ is not hydrogen.

In some embodiments, R⁵ is optionally substituted C₁-C₆ alkyl, F, Cl, Br, OCH₃, OCH₂CH₃, or CN; and R⁶ is H.

In some embodiments, R⁵ is H; and R⁶ is optionally substituted C₁-C₆ alkyl, F, Cl, Br, OCH₃, OCH₂CH₃, or CN.

Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34(1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

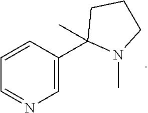

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49(1), 161-166; Wang et al., Drug Dev Res 1998, 45(1), 10-16; Secor et al., Tetrahedron Lett. (1981), 22(33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

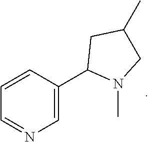

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

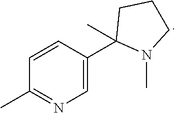

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

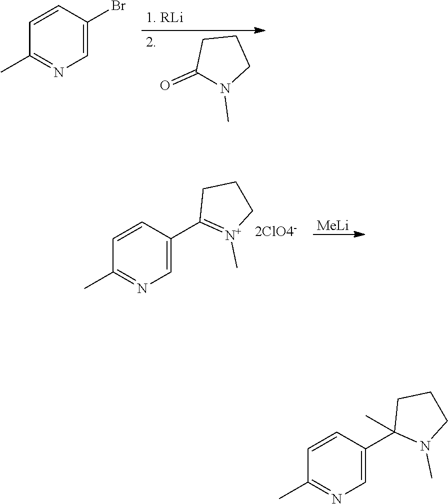

In some embodiments, R⁵ is H or CH₃, R⁶ is H, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from the group consisting of:

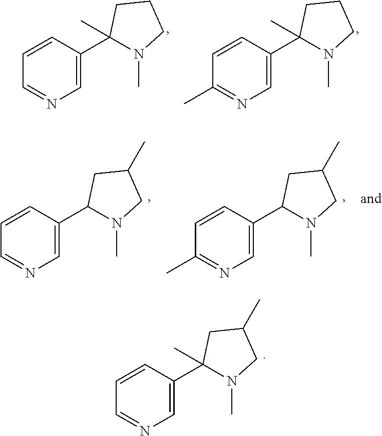

In some embodiments, R⁵ is H, R⁶ is F, CH₃, or OCH₃, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from the group consisting of:

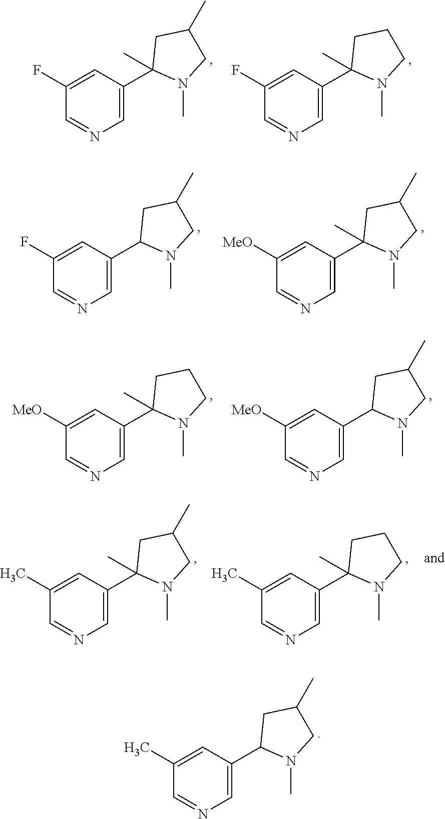

A substituted 3-(1-methylpyrrolidin-2-yl)pyridine of Formula II as described herein may be present as a single enantiomer or as a mixture of enantiomers. In some embodiments, the substituted 3-(1-methylpyrrolidin-2-yl)pyridine of Formula II is present in racemic form, meaning there are equal amounts of (R)- and (S)-enantiomers present. In some embodiments, the oral composition or product comprises unequal amounts of (R)- and (S)-enantiomer (i.e., is enriched in either the (R)- or (S)-enantiomer). In some embodiments, the oral composition or product predominantly comprises the substituted 3-(1-methylpyrrolidin-2-yl)pyridine of Formula II in the (R)-configuration, for example, about 90% or more of the total quantity of substituted 3-(1-methylpyrrolidin-2-yl)pyridine present is in the (R)-configuration. In some embodiments, the oral composition or product predominantly comprises the substituted 3-(1-methylpyrrolidin-2-yl)pyridine of Formula II in the (S)-configuration, for example, about 90% or more of the total quantity of substituted 3-(1-methylpyrrolidin-2-yl)pyridine present is in the (S)-configuration. In some embodiments, the oral composition or product comprises 95% or more of the (S)-configuration of the substituted 3-(1-methylpyrrolidin-2-yl)pyridine of Formula II, based on the total amount of substituted 3-(1-methylpyrrolidin-2-yl)pyridine present.

In some embodiments, the substituted 3-(1-methylpyrrolidin-2-yl)pyridine of Formula II is non-racemic, and has one of the following structures:

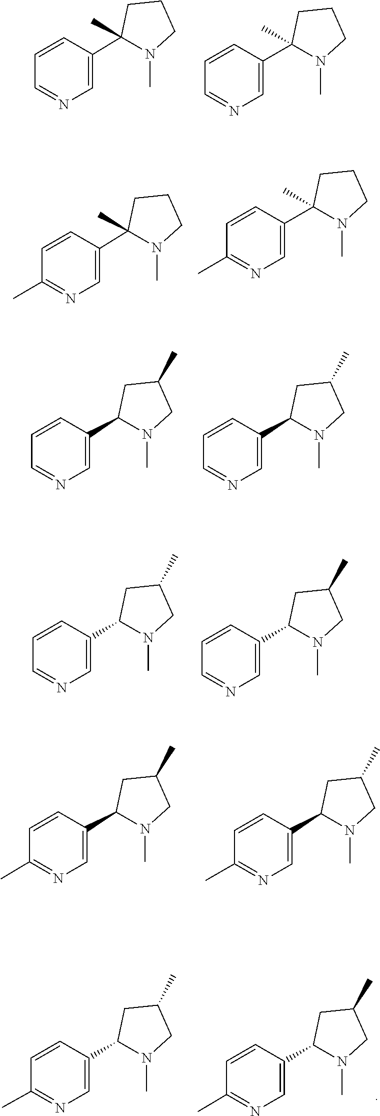

In some embodiments, the substituted 3-(1-methylpyrrolidin-2-yl)pyridine of Formula II is non-racemic, and has a structure selected from the group consisting of:

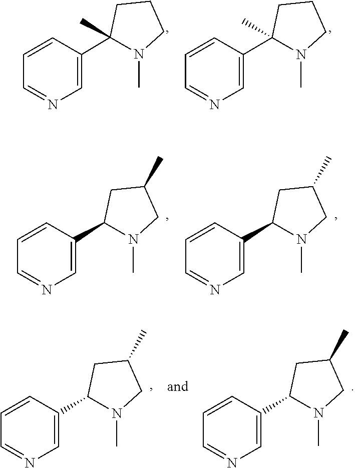

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines of Formula II such as those described herein has been reported in, for example, Lin et al., J. Med. Chem., 1994, 37, 3542-3553; Dukat et al. European Journal of Medicinal Chemistry, 31(11), 1996, 875-888; U.S. Pat. No. 9,440,948; Wang et al., Drug Dev Res 1998, 45(1), 10-16 (each of which is incorporated herein by reference), among many others. Generally, small substituents such as methyl groups are well tolerated at the 2′- or 4′-positions of the nicotine pyrrolidine ring, and small substituents such as alkyl, halogen, alkoxy, and the like are well tolerated at the 5- or 6-position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and is believed that certain alkyl substitutions for hydrogen on the pyridine and/or pyrrolidine rings of the substituted 3-(1-methylpyrrolidin-2-yl)pyridine compound preserve the general pharmacological Wang et al., Drug Dev Res 1998, 45(1), 10-16. Without wishing to be bound by any particular theory, these compounds are believed to provide the general pharmacological profile and physiological effects of nicotine while offering the potential for one or more of greater potency, reduced product consumption, more rapid and/or complete absorption, and the like. Particularly, it is believed that in some embodiments, substituted 3-(1-methylpyrrolidin-2-yl)pyridines of the disclosure according to Formula II are readily absorbed through oral mucosa by virtue of their lipophilicity.

In some embodiments, the substituted 3-(1-methylpyrrolidin-2-yl)pyridine of Formula II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula II has a calculated log P from about 1.2 to about 1.7.

In some embodiments, the substituted 3-(1-methylpyrrolidin-2-yl)pyridine of Formula II has the structure:

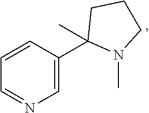

having a potency factor of about 1.35, meaning that embodiments, the amount of substituted 3-(1-methylpyrrolidin-2-yl)pyridine present may be about 74% of the amount of nicotine required to achieve the same effect.

In some embodiments, the substituted 3-(1-methylpyrrolidin-2-yl)pyridine of Formula II has the structure:

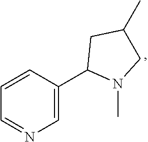

having a potency factor of about 0.9, meaning that in some embodiments, the amount of substituted 3-(1-methylpyrrolidin-2-yl)pyridine present may be about 110% of the amount of nicotine required to achieve the same effect.

The substituted 3-(1-methylpyrrolidin-2-yl)pyridine of Formula II may be present in the oral composition or product as the free base, as a salt with a suitable acid, or in the form of an ion pair with an organic acid. Each of these forms is described further herein below.

### 3-(azetidin-2-yl)pyridines and 3-(azetidin-2-ylmethoxy)pyridines

As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

As described above, some products of the disclosure do not contain nicotine and do not contain any compounds obtained by chemical reactions utilizing nicotine as a starting material.

In some embodiments, the 3-(azetidin-2-yl)pyridine or 3-(azetidin-2-ylmethoxy)pyridine has a structure according to Formula III:

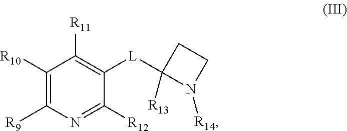

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

In some embodiments, L is a bond.

In some embodiments, R⁹ is CH₃, F, Cl, Br, OCH₃, OEt, or CN.

In some embodiments, R⁹ is H or CH₃; and R⁶, R⁷, and R⁸ are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-yl) pyridine is 3-(azetidin-2-yl)pyridine, and has a structure:

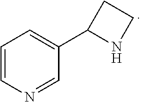

The compound 3-(azetidin-2-yl)pyridine is known in the literature. The synthesis of this compound has been reported in JOC 1979, 44(18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl) pyridine is 3-(1-methylazetidin-2-yl)pyridine, having the structure:

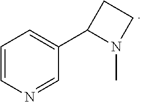

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl) pyridine has a structure selected from the group consisting of:

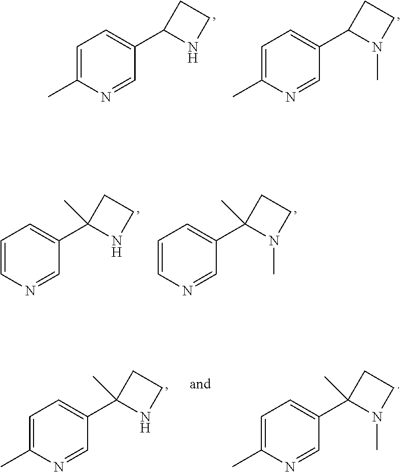

Such compounds are either known, or may be readily prepared according to adaptations of methods utilized for preparation of related 3-(azetidin-2-yl) pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the resinate complexes provided herein comprise a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-ylmethoxy)pyridine has a structure selected from the group consisting of:

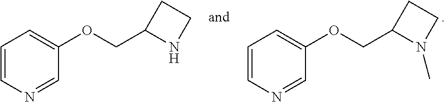

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

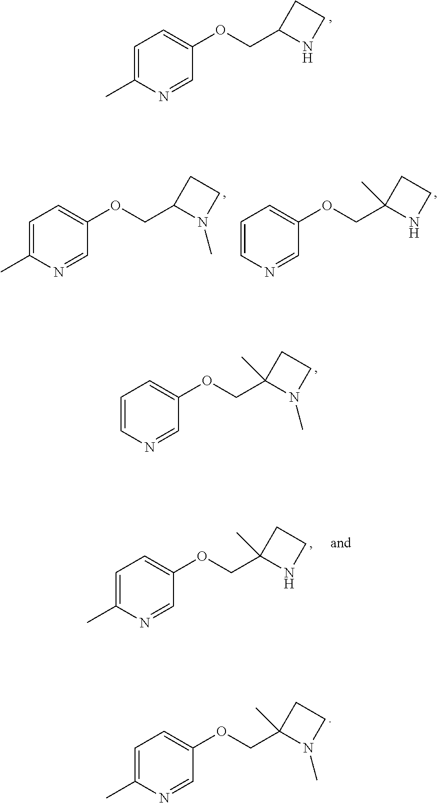

Such compounds are either known, or may be readily prepared according to adaptations of methods utilized for preparation of the 3-(azetidin-2-ylmethoxy)pyridines, the 3-(azetidin-2-yl)pyridines, and substituted 3-(1-methylpyrrolidin-2-yl)pyridines described herein above.

A 3-(azetidin-2-yl)pyridine or 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the oral composition or product of the disclosure comprises unequal amounts of (R)- and (S)-enantiomer (i.e., is enriched in either the (R)- or (S)-enantiomer). In some embodiments, the oral composition or product of the disclosure predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the oral composition or product of the disclosure predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the oral composition or product of the disclosure comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

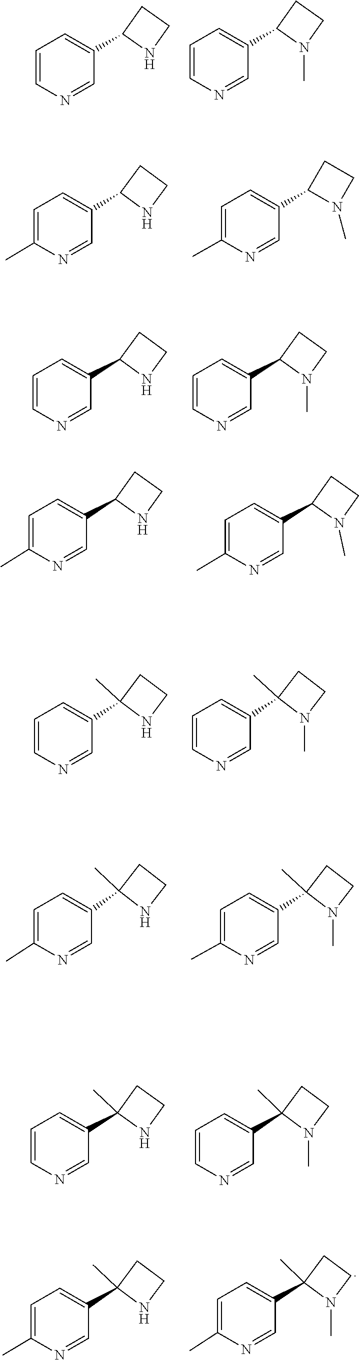

In some embodiments, the optionally substituted 3-(azetidin-2-ylmethoxy)pyri dine is non-racemic, and has one of the following structures:

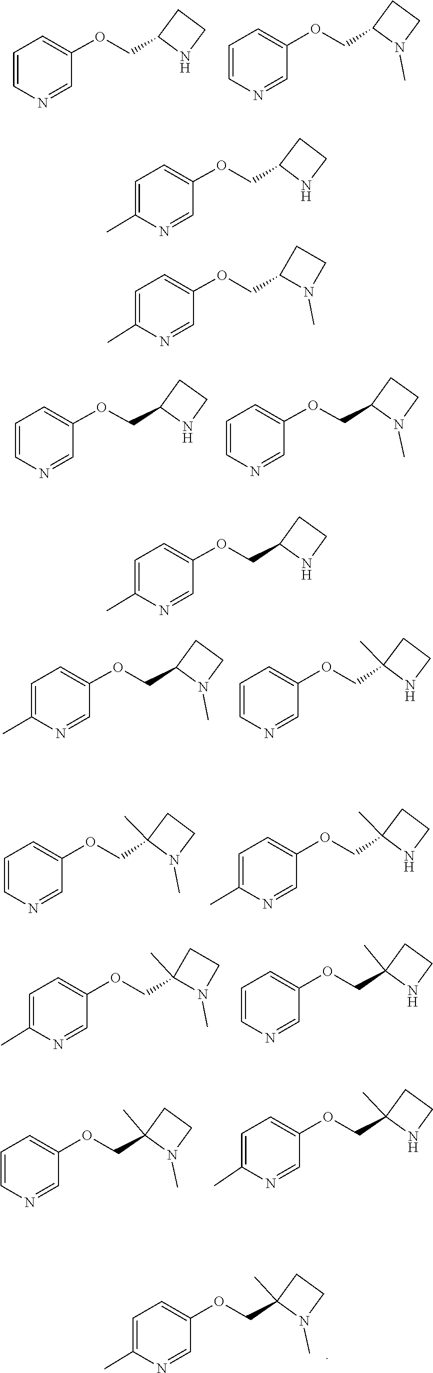

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the α4β2 subtype. The overall pharmacological profiles have been shown to be or are expected to be comparable to that of nicotine.

In some embodiments, the optionally substituted 3-(azetidin-2-yl)pyridine has the structure:

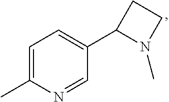

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

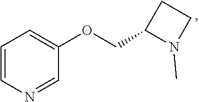

having a potency factor of about 3, meaning that in some embodiments, the amount of 3-(azetidin-2-ylmethoxy)pyridine present may be about 33% of the amount of nicotine required to achieve the same effect.

### Other Active Ingredients

In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine of the present disclosure is replaced with, or combined with, other active ingredients that provide the same general pharmacological profile and/or physiological effects of nicotine. Certain of these active ingredients may be equipotent or even more potent than nicotine with respect to binding affinity to the nicotinic acetylcholine receptor, and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example active ingredients of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other active ingredient is cytisine or an analog thereof. Cytisine is a naturally occurring alkaloid present in certain plant genera, such as Laburnum and Cytisus of the family Fabaceae. Cytisine (CAS Registry No. 485-35-8) has the structure:

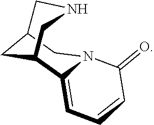

Cytisine is commercially available and has been utilized in post-Soviet states for more than 40 years as an aid to smoking cessation under the brand name Tabex (Sopharma AD). Cytisine is a partial agonist of the α4β2 nicotinic acetylcholine receptor.

In some embodiments, the optional other active ingredient is varenicline or an analog thereof. Varenicline is commercially available as Chantix® (Pfizer) and is a medication used as an aid for smoking cessation. Varenicline (CAS Registry No. 249296-44-4) has the structure:

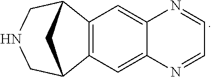 [SMILES: c1cnc2cc3c(cc2n1)[C@@H]1CNC[C@H]3C1]

Like cytisine, varenicline is a partial agonist of the α4β2 nicotinic acetylcholine receptor.

The quantity of the other active ingredient optionally present in the oral compositions and products disclosed herein may vary. Typically, the optional other active ingredient, calculated as the free base, is present in a concentration of at least about 0.001% by weight of the oral composition or product, such as in a range from about 0.01% to about 10%. In some embodiments, the other active ingredient is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the oral composition or product. In some embodiments, the other active ingredient is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the oral composition or product. One of skill in the art will recognize that the amount of any particular other active ingredient present in composition may vary based on the potency of the compound, the other components of the oral composition or product, and the desired physiological effect for the oral composition or product.

The optional other active ingredient may be present in the composition as the free base, as a salt with a suitable acid, or in the form of an ion pair with an organic acid. Each of these forms is described further herein below.

### Resin Complex

As described herein above, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a resinate complex, where the referenced compound is associated with the resin base component as described herein above.

In some embodiments, all of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a resinate complex. In some embodiments, a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a resinate complex, and a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in a different form, e.g., as described herein below.

A resin base material generally comprises a given number of acidic sites. Within a resinate complex as provided herein, the acidic sites originally present on the resin base material comprise a first potion of the acidic sites that are associated with ions of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient and a second portion of the acidic sites that are associated with protons (H+) The ratio of the first portion of acidic sites to the second portion of acidic sites (i.e., the ratio of ions of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient to protons associated with the polymer) can vary. In some embodiments, at least 1%, at least 2%, at least 5%, at least 10%, at least 15%, at least 20%, at least 30%, at least 40%, at least 50%, at least 60%, at least 70%, at least 80%, or at least 90% of the acidic sites on the resinate base material are associated with ions of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient. For example, in some embodiments, 1% to 90%, 2% to 80%, 5% to 60%, or 10% to 50% of the acidic sites on the resinate base material are associated with ions of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient in the final resinate complex.

As such, the resinate complexes described herein can have varying amounts of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient associated therewith. For example, in some embodiments, a resinate complex is provided, comprising about 0.5% by weight or more, about 1% by weight or more, about 2% by weight or more, about 3% by weight or more, about 4% by weight or more, or about 5% by weight or more of substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, based on the total dry weight of the resinate complex. For example, in some embodiments, the resinate complexes provided herein comprise about 0.5% to about 10% by weight, about 1% by weight to about 8% by weight, about 2% by weight to about 6% by weight of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient.

The method by which the disclosed resinate complexes are prepared can vary. For example, methods for the preparation of nicotine-resinate complexes are known and can be modified for the preparation of resinate complexes according to the present disclosure. Certain non-limiting disclosures relating to methods of preparing nicotine-resinate complexes are found in U.S. Pat. No. 8,986,668 to Andersen/Fertin Pharma A/S and U.S. Pat. No. 6,586,449 to Walling/Cambrex Charles Cit, Inc., which are incorporated herein by reference in their entireties. Briefly, in some embodiments, the resinate base material (typically in acid form) and the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient are combined within a liquid, e.g., including, but not limited to, water (which can optionally further comprise a polyol).

In some embodiments, the resinate complex is produced by treating a resinate base material to reduce the acidity thereof and then treating the proton-displaced polymer with the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient. In some embodiments, the resinate complex is provided by treating a salt-form resinate base material with the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient. Such methods pertaining to the preparation of nicotine-containing resins are provided in European Patent Application 24180525.8, which is incorporated herein by reference in its entirety and can be suitably adapted for purposes of the present disclosure to associate substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient with a resin to form the resinate complexes described herein.

In some embodiments, the as-prepared resinate complex is further processed, e.g., by drying and/or separating it from the liquid in which it was prepared, e.g., by filtration (such as vacuum filtration) or the like. In some embodiments, the resinate complex is washed, e.g., with a solvent (such as water). In some embodiments, multiple washing steps can be conducted. Drying can be done, in some embodiments, in an oven (e.g., convection oven) at elevated temperature (e.g., above 20 °C, such as 20 °C to 60 °C). The time for which the material is dried can be that time sufficient to achieve the desired moisture content.

### Other Forms

It is understood that the association of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient with the resin as provided herein will vary, depending upon the surrounding environment of the resinate complex, e.g., the pH value(s) to which it is exposed and the presence of certain cations (e.g., cations of calcium, sodium, and the like). Typically, as noted above, at least some of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient originally associated with the resin base will be released from the resin base within the oral composition into which it is incorporated (referred to herein below as “released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient”). Such released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient can be present in one or more forms, including free base form, salt form, co-crystal form, salt co-crystal form, and ion pair form, as briefly described herein below.

Free base. In some embodiments, at least a portion of released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of its free base. In some embodiments, released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in free base form and is adsorbed in/on a carrier such as a microcrystalline cellulose material to form an adsorption complex.

Salt. In some embodiments, at least a portion of released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a salt. A “salt” of such compounds is a form with fixed stoichiometry, characterized by interaction between the compound in ionic form and a coformer in ionic form (e.g., an acid) via the transfer of one or more protons from the coformer donor to the compound acceptor. Stoichiometry can vary and hydrates and other solvates of salts are further contemplated herein.

The structures of substituted 3-(1-methylpyrrolidin-2-yl)pyridine, optionally substituted 3-(azetidin-2-yl)pyridine, and optionally substituted 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient can be provided using the types of ingredients and techniques set forth for nicotine in U.S. Pat. No. 2,033,909 to Cox et al. and Perfetti, Beitrage Tabakforschung Int., 12: 43-54 (1983), which are incorporated herein by reference.

Such salt forms can be provided where the oral composition into which the resinate complex is added comprises suitable coformers, e.g., suitable acids. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexenoic acid, trimesic acid, 5-nitroisophthalic acid, and zinc chloride monohydrate (forming a hydrated zinc chloride complex salt). In some embodiments, acids optionally included within the disclosed oral compositions are selected to give better taste sensation, as it has been found that salts of certain organic acids, such as galactaric acid, offer a better taste sensation relative to salts of acids such as tartaric or phthalic acids.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified. In some embodiments, released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient present in the form of a salt advantageously has a relatively low log D, indicative of good water solubility (e.g., from about −1.0 to about 3 at a pH in a range from about 3 to about 11, such as from about −0.5 to about 2, about −0.3 to about 1, or about −0.1 to about 0). Without wishing to be bound by theory, it is believed that highly water-soluble salt forms may exhibit a high rate of dissolution, which may be favorable in certain embodiments.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a salt with succinic acid or galactaric acid.

Salts as described herein can, in some embodiments, exist in various polymorphic and pseudopolymorphic forms. Polymorphism is the ability of a crystalline material to exist in more than one form or crystal structure. Polymorphism can result., e.g., from the existence of different crystal packing structures (packing polymorphism) or from the existence of different conformers of the same molecule (conformational polymorphism). Pseudopolymorphism is the result of hydration or solvation of a material and is also referred to as solvomorphism.

Co-crystal. In some embodiments, at least a portion of released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient in combination with an organic and/or an inorganic coformer.

Examples of suitable coformers for co-crystals include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (β-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids. In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

Salt co-crystal. In some embodiments, the released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, and wherein a second coformer does not transfer a proton to the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient. Suitable acids and coformers are generally those described herein above with respect to salts and co-crystals.

The stoichiometry of co-crystals and salt co-crystals as described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient and to one another can also vary. The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of a salt-co-crystal. In some embodiments, the salt-co-crystal is a bis-orotic acid salt-co-crystal. In some embodiments, the bis-orotic acid salt-co-crystal is a hemi-hydrate.

Ion Pairs. In some embodiments, at least a portion of released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the composition as disclosed herein, and/or upon oral use of the oral composition or product (e.g., upon contact with saliva), the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed oral composition or product, both before and during use by the consumer, may vary based on, for example, pH, the nature of the acid, the concentration of released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, the concentration of the acid or conjugate base of the acid present in the oral composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient through the oral mucosa. However, as described above, in some embodiments, the properties of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient are such that no ion pairing is required, and accordingly, the composition is substantially or completely free of any ion pairing. By “substantially free” it is meant that no measurable degree of ion pairing is present.

In embodiments where ion pairing is desired, the oral composition or product comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the oral composition or product of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the oral composition or product may be characterized as substantially or completely free of organic acids (i.e., having less than 0.001% by weight of organic acid, or less than 0.0001%, or even 0% by weight of organic acid, based on the total weight of the composition, or as having an amount of organic acid below the limit of detection). This is not to be interpreted as meaning that the oral composition or product is substantially or completely free of substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient co-crystals unless explicitly recited.

### Organic Acid

In embodiments where ion pairing of released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is desired, the oral composition or product comprises an organic acid as defined herein above, and/or an alkali metal salt thereof. Suitable organic acids for ion pairing will typically have a range of lipophilicities (i.e., a polarity giving an appropriate balance of water and organic solubility). Typically, lipophilicities of suitable organic acids, as indicated by log P, will vary between about 0 and about 12 (more soluble in octanol than in water). In some embodiments, the organic acid has a log P value from about 0 to about 12, e.g., from about 0.5, 1.0. about 1.5, about 2.0, about 2.5, about 3.0, about 3.5, about 4.0, about 4.5, about 5.0, about 5.5, about 6.0, about 6.5, about 7.0, about 7.5, or about 8.0, to about 8.5, about 9.0, about 9.5, about 10.0, about 10.5, about 11.0, about 11.5, or about 12.0.

Without wishing to be bound by theory, it is believed that moderately lipophilic organic acids (e.g., log P of from about 1.4 to about 4.5) produce ion pairs which are of a polarity providing good octanol-water partitioning of the ion pair, and hence partitioning of substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, into octanol versus water. As discussed above, such partitioning into octanol is predictive of favorable oral availability.

In some embodiments, the organic acid for ion pairing has a log P value from about 3.0 to about 8.0, about 10.0, or even 12.0. In some embodiments, the presence of certain solvents or solubilizing agents (e.g., inclusion in the oral composition or product of glycerin or propylene glycol) may be beneficial in solubilizing organic acids and the corresponding salts or ion pairs thereof for highly lipophilic organic acids (e.g., higher than about 4.5).

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g., nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, —NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid for ion pairing is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid for ion pairing is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid for ion pairing may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid for ion pairing may include more than one carboxylic acid group and one or more hydroxyl groups. Non-limiting examples of such acids include tartaric acid, citric acid, and the like.

In some embodiments, the organic acid for ion pairing is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful for ion pairing in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids for ion pairing include, but are not limited to, the list of organic acids in Table 1.

TABLE 1
Non-limiting examples of suitable organic acids for ion pairing

| Acid Name | log(P)* |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-(4-isobutylphenyl)propanoic acid | 3.5 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptanesulfonic acid | 2.0 |
| 1-octanesulfonic acid | 2.5 |
| 1-nonanesulfonic acid | 3.1 |
| monooctyl succinate | 2.8 |
| tocopherol succinate | 10.2 |
| monomenthyl succinate | 3 |
| monomenthyl glutarate | 3.4 |
| norbixin ((2E,4E,6E,8E,10E,12E,14E,16E,18E)-4,8,13,17- | 7.2 |
| tetramethylicosa-2,4,6,8,10,12,14,16,18-nonaenedioic | |
| acid) | |
| bixin ((2E,4E,6E,8E,10E,12E,14E,16Z,18E)-20-methoxy- | 7.5 |
| 4,8,13,17-tetramethyl-20-oxoicosa-2,4,6,8,10,12,14,16,18- | |
| nonaenoic acid | |

*Values obtained from PubChem or calculated

The selection of organic acid for ion pairing may further depend on additional properties in addition to consideration of the log P value. For example, an organic acid should be one recognized as safe for human consumption, and which has acceptable flavor, odor, volatility, stability, and the like. Determination of appropriate organic acids is within the purview of one of skill in the art.

In some embodiments, the organic acid for ion pairing is a mono ester of a dicarboxylic acid or a poly-carboxylic acid. In some embodiments, the dicarboxylic acid is malonic acid, succinic acid, glutaric acid, adipic acid, fumaric acid, maleic acid, or a combination thereof. In some embodiments, the dicarboxylic acid is succinic acid, glutaric acid, fumaric acid, maleic acid, or a combination thereof. In some embodiments, the dicarboxylic acid is succinic acid, glutaric acid, or a combination thereof.

In some embodiments, the alcohol forming the mono ester of the dicarboxylic acid is a lipophilic alcohol. Examples of suitable lipophilic alcohols include, but are not limited to, octanol, menthol, and tocopherol. In some embodiments, the organic acid is an octyl mono ester of a dicarboxylic acid, such as monooctyl succinate, monooctyl fumarate, or the like. In some embodiments, the organic acid is a monomenthyl ester of a dicarboxylic acid. Certain menthyl esters may be desirable in the oral composition or product as described herein by virtue of the cooling sensation they may provide upon use of the product comprising the composition. In some embodiments, the organic acid is monomenthyl succinate, monomenthyl fumarate, monomenthyl glutarate, or a combination thereof. In some embodiments, the organic acid is a monotocopheryl ester of a dicarboxylic acid. Certain tocopheryl esters may be desirable in the oral composition or product as described herein by virtue of the antioxidant effects they may provide. In some embodiments, the organic acid is tocopheryl succinate, tocopheryl fumarate, tocopheryl glutarate, or a combination thereof.

In some embodiments, the organic acid for ion pairing is a carotenoid derivative having one or more carboxylic acids. Carotenoids are tetraterpenes, meaning that they are produced from 8 isoprene molecules and contain 40 carbon atoms. Accordingly, they are usually lipophilic due to the presence of long unsaturated aliphatic chains, and are generally yellow, orange, or red in color. Certain carotenoid derivatives can be advantageous in in the disclosed oral composition or product by virtue of providing both ion pairing and serving as a colorant in the materials and articles. In some embodiments, the organic acid is 2E,4E,6E,8E,10E,12E,14E,16Z,18E)-20-methoxy-4,8,13,17-tetramethyl-20-oxoicosa-2,4,6,8,10,12,14,16,18-nonaenoic acid (bixin) or an isomer thereof. Bixin is an apocarotenoid found in annatto seeds from the achiote tree (Bixa orellana) and is the naturally occurring pigment providing the reddish orange color to annatto. Bixin is soluble in fats and alcohols but insoluble in water, and is chemically unstable when isolated, converting via isomerization into the double bond isomer, trans-bixin (β-bixin), having the structure:

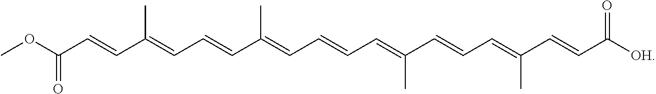 [SMILES: COC(=O)/C=C/C(C)=C/C=C/C(C)=C/C=C/C=C(C)/C=C/C=C(C)/C=C/C(=O)O]

In some embodiments, the organic acid for ion pairing is (2E,4E,6E,8E,10E,12E,14E,16E,18E)-4,8,13,17-tetramethylicosa-2,4,6,8,10,12,14,16,18-nonaenedioic acid (norbixin), a water-soluble hydrolysis product of bixin having the structure:

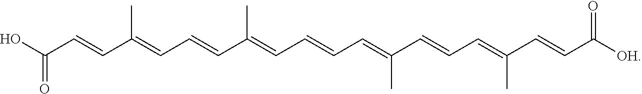 [SMILES: CC(/C=C/C=C(C)/C=C/C(=O)O)=C\C=C\C=C(C)\C=C\C=C(C)\C=C\C(=O)O]

In some embodiments, more than one organic acid for ion pairing may be present. For example, the oral composition or product may comprise two, or three, or four, or more organic acids for ion pairing. Accordingly, reference herein to “an organic acid” contemplates mixtures of two or more organic acids. The relative amounts of the multiple organic acids may vary. For example, an oral composition or product may comprise equal amounts of two, or three, or more organic acids, or may comprise different relative amounts. In this manner, it is possible to include certain organic acids (e.g., citric acid or myristic acid) which have a log P value outside the desired range, when combined with other organic acids to provide the desired average log P range for the combination. In some embodiments, it may be desirable to include organic acids in the oral composition or product for ion pairing which have log P values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the composition remains below the threshold which would be found objectionable from a sensory perspective.

In some embodiments, the disclosed oral composition or product comprises an organic acid for ion pairing which is a monoester of a dicarboxylic acid or is a carotenoid derivative having one or more carboxylic acids as described herein above, and further comprises an additional organic acid or salt thereof. In some embodiments, the additional organic acid is benzoic acid, an alkali metal salt thereof, or a combination thereof.

In some embodiments, the disclosed oral composition or product comprises an alkali metal salt of an organic acid. For example, at least a portion of the organic acid may be present in the composition in the form of an alkali metal salt. Suitable alkali metal salts include lithium, sodium, and potassium. In some embodiments, the alkali metal is sodium or potassium. In some embodiments, the alkali metal is sodium. In some embodiments, the oral composition or product comprises an organic acid and a sodium salt of the organic acid.

In some embodiments, the molar ratio of the organic acid to the sodium salt (or other alkali metal) of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the composition, wherein the organic acid is added in excess of the sodium salt, in equimolar quantities with the sodium salt, or as a fraction of the sodium salt. One of skill in the art will recognize that the relative amounts will be determined by the desired pH of the composition, as well as the desired ionic strength. For example, the organic acid may be added in a quantity to provide a desired pH level of the composition, while the alkali metal (e.g., sodium) salt is added in a quantity to provide the desired extent of ion pairing. As one of skill in the art will understand, the quantity of organic acid (i.e., the protonated form) present in the composition, relative to the alkali metal salt or conjugate base form present in the composition, will vary according to the pH of the composition and the pKa of the organic acid, as well as according to the actual relative quantities initially added to the oral composition or product.

The amount of organic acid or alkali metal salt thereof present in the oral composition or product, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, or other active ingredient that is ion paired with the organic acid increases. This typically increases the partitioning of the released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient in the form of an ion pair, into octanol versus water as measured by the log P (the log₁₀ of the partitioning coefficient). In some embodiments, the oral composition or product comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient, calculated as the free base of the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient.

In some embodiments, the disclosed oral composition or product comprises from about 2 to about 10, or from about 2 to about 5 molar equivalents of the organic acid, the alkali metal salt thereof, or the combination thereof, relative to the anticipated released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient on a free-base basis. The anticipated released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient can be calculated, e.g., based on the Henderson-Hasselbalch equation. In some embodiments, the organic acid, the alkali metal salt thereof, or the combination thereof, is present in a molar ratio with the anticipated released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the oral composition or product comprises benzoic acid and sodium benzoate wherein a total amount of benzoate (i.e., benzoic acid and benzoate) is in a molar ratio in a range from about 3 to about 5 relative to the anticipated released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient. In some embodiments, the molar ratio of the total amount of benzoate to the anticipated released substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient is about 3.2 or about 4.8.

In some embodiments, the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. Reference herein to “a composition pH” means the pH of an aqueous solution of the oral composition or product prepared by dissolving or suspending 5 grams of the oral composition or product in 95 grams of water and measuring the pH of the resulting solution with a calibrated pH meter.

In some embodiments, the organic acid inclusion is sufficient to provide a composition pH of from about 4.5 to about 6.5, for example, from about 4.5, about 5.0, or about 5.5, to about 6.0, or about 6.5. In some embodiments, the desired composition pH is from about 4.5 to about 6.5, and the organic acid is provided in a quantity sufficient to provide such a pH. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the composition to the desired value. In some embodiments, a buffer (e.g., a buffer as described herein below) is added to the composition to the desired value, and/or to maintain the pH of the composition at the desired value.

In some embodiments, the oral composition further comprises a solubility enhancer to increase the solubility of one or more of the organic acid or salt thereof. Suitable solubility enhancers include, but are not limited to, humectants as described herein, such as glycerol or propylene glycol.

### Oral Compositions and Products

The present disclosure provides the resinate complexes, and also compositions and products configured for oral use comprising such resinate complexes. The compositions and products may be provided in a variety of forms. Accordingly, a product may include the composition itself or the composition positioned within a unitizing structure, such as a pouch or the like.

The term “configured for oral use” as used herein means that the composition or product is provided in a form such that during use, saliva in the mouth of the user causes one or more of the components of the composition (e.g., basic amine, flavoring agents and/or active ingredients) to pass into the mouth of the user. In certain embodiments, the composition or product is adapted to deliver components to a user through mucous membranes in the user's mouth, the user's digestive system, or both, and, in some instances, said component is a nicotine component or an active ingredient (including, but not limited to, for example, nicotine, a stimulant, vitamin, amino acid, botanical, or a combination thereof) that can be absorbed through the mucous membranes in the mouth or absorbed through the digestive tract when the product is used.

Compositions and products configured for oral use as described herein may take various forms, including gels, pastilles, gums, chews, melts, tablets, lozenges, granules, powders, and pouches. Gels can be soft or hard. Certain compositions of the disclosure are in the form of solids. Certain compositions can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

The compositions and products of the present disclosure may be dissolvable. As used herein, the terms “dissolve,” “dissolving,” and “dissolvable” refer to compositions having aqueous-soluble components that interact with moisture in the oral cavity and enter into solution, thereby causing gradual consumption of the composition. According to one aspect, the dissolvable composition is capable of lasting in the user's mouth for a given period of time until it completely dissolves. Dissolution rates can vary over a wide range, from about 1 minute or less to about 60 minutes. For example, fast release compositions typically dissolve and/or release the desired component(s) (e.g., active ingredient, flavor, and the like) in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the composition. In other embodiments, the products do not dissolve during the product's residence in the user's mouth.

The compositions as disclosed herein can be formed into a variety of shapes, including pills, tablets, spheres, strips, films, sheets, coins, cubes, beads, ovoids, obloids, cylinders, bean-shaped, sticks, or rods. Cross-sectional shapes of the composition can vary, and example cross-sectional shapes include circles, squares, ovals, rectangles, and the like. Such shapes can be formed in a variety of manners using equipment such as moving belts, nips, extruders, granulation devices, compaction devices, and the like.

Certain compositions configured for oral use are in the form of pastilles. As used herein, the term “pastille” refers to a dissolvable oral composition made by solidifying a liquid or gel composition so that the final composition is a somewhat hardened solid gel. The rigidity of the gel is highly variable. A pastille product may alternatively be referred to as a soft lozenge. In certain embodiments, the pastille products of the disclosure are characterized by sufficient cohesiveness to withstand light chewing action in the oral cavity without rapidly disintegrating. The pastille products of the disclosure typically do not exhibit a highly deformable chewing quality as found in conventional chewing gum.

In some embodiments, the products disclosed herein may be in the form of a dissolvable lozenge product configured for oral use. Example lozenge-type products of the disclosure have the form of a lozenge, tablet, microtab, or other tablet-type product. See, for example, the types of nicotine-containing lozenges, lozenge formulations, lozenge formats and configurations, lozenge characteristics and techniques for formulating or manufacturing lozenges set forth in U.S. Pat. No. 4,967,773 to Shaw; U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al.; U.S. Pat. No. 6,248,760 to Wilhelmsen; and 7,374,779; US Pat. Pub. Nos. 2001/0016593 to Wilhelmsen; 2004/0101543 to Liu et al.; 2006/0120974 to Mcneight; 2008/0020050 to Chau et al.; 2009/0081291 to Gin et al.; and 2010/0004294 to Axelsson et al.; which are incorporated herein by reference.

Lozenge products are generally described as “hard” and are distinguished in this manner from soft lozenges (i.e., pastilles). Hard lozenges are mixtures of sugars, sugar alcohols, and/or carbohydrates in an amorphous state (e.g., present in some embodiments in an amount of greater than about 80% by weight). Although they are made from aqueous syrups, the water, which is initially present, evaporates as the syrup is boiled during processing so that the moisture content in the finished product is very low, such as 0.5% to 1.5% by weight. To obtain lozenges that are hard and not tacky, the temperature of the melt generally must reach the hard crack stage, with an example temperature range of 149° to 154 °C.

In some embodiments, oral products provided herein may be in the form of center-filled pastilles or lozenges, for example, such that the interior (or at least a portion) of the product has one or more different organoleptic properties (e.g., texture, mouthfeel, taste, etc.) from the outer surface thereof (or other portion thereof). Such center-filled pastille or lozenge formulations may include a liquid and/or a gel and/or a meltable and/or a chewable and/or a gummy and/or an effervescent center-filling that is surrounded by a harder outer shell that can be associated with pastille-type or lozenge products as described herein. In such embodiments, the center-filling may be described as having less rigidity and/or increased softness compared to the outer shell. In some embodiments, the center-filling may or may not include an active ingredient therein. For example, in some embodiments, both the outer shell and the center-filling formulations may include an active ingredient so as to provide an extended release of the active ingredient therefrom. In some embodiments, at least the outer shell formulation includes a pastille formulation as described herein above. In other embodiments, both the outer shell formulation and the center-filling formulation may comprise a pastille formulation as described herein having similar or different organoleptic properties.

In some embodiments, the composition can be chewable, meaning the composition has a mild resilience or “bounce” upon chewing, and possesses a desirable degree of malleability. A composition in chewable form may be entirely dissolving or may be in the form of a non-dissolving gum in which only certain components (e.g., active ingredients, flavor, sweetener) dissolve, leaving behind a non-dissolving matrix. Chewable embodiments generally include a binder, such as a natural gum or pectin. In some embodiments, the composition in chewable form comprises pectin and an organic acid, along with one or more sugar alcohols in an amount by weight of at least 50%, based on the total weight of the composition. Generally, the pectin is present in an amount of from about 1 to about 3% by weight, based on the total weight of the composition.

In some embodiments, the composition can be meltable as discussed, for example, in U.S. Pat. No. 11,116,237 to Cantrell et al., incorporated by reference herein in its entirety. As used herein, “melt,” “melting,” and “meltable” refer to the ability of the composition to change from a solid state to a liquid state. That is, melting occurs when a substance (e.g., a composition as disclosed herein) changes from solid to liquid, usually by the application of heat. The application of heat in regard to a composition as disclosed herein is provided by the internal temperature of a user's mouth. Thus, the term “meltable” refers to a composition that is capable of liquefying in the mouth of the user as the composition changes phase from solid to liquid and is intended to distinguish compositions that merely disintegrate in the oral cavity through loss of cohesiveness within the composition that merely dissolve in the oral cavity as aqueous-soluble components of the composition interact with moisture. Generally, meltable compositions comprise a lipid as described herein. In some embodiments, the composition in meltable form comprises a lipid in an amount of from about 35 to about 50% by weight, based on the total weight of the composition, and a sugar alcohol in an amount of from about 35 to about 55% by weight, based on the total weight of the composition. In some embodiments, the sugar alcohol is isomalt, erythritol, sorbitol, arabitol, ribitol, maltitol, dulcitol, iditol, mannitol, xylitol, lactitol, or a combination thereof. In some embodiments, the sugar alcohol is isomalt.

In certain embodiments, the composition is in the form of a compressed or molded pellet. Example pellet weights range from about 250 mg to about 1500 mg, such as about 250 mg to about 700 mg, or from about 700 mg to about 1500 mg. The pellet can have any of a variety of shapes, including traditional pill or tablet shapes. Generally, the composition in tablet form comprises a glucose-polysaccharide blend and a sugar alcohol. In some embodiments, the glucose-polysaccharide blend is present in an amount of from about 35 to about 50% by weight, based on the total weight of the composition; and the sugar alcohol is present in an amount of from about 30 to about 45% by weight, based on the total weight of the composition. In some embodiments, the sugar alcohol is isomalt, erythritol, sorbitol, arabitol, ribitol, maltitol, dulcitol, iditol, mannitol, xylitol, lactitol, or a combination thereof. In some embodiments, the sugar alcohol is isomalt.

In one embodiment, an oral composition comprising a resinate complex as described herein is disposed within a moisture-permeable container (e.g., a water-permeable pouch). The composition enclosed in the pouch may be in any desired form. In certain embodiments, the composition is in granular form. Such compositions in the water-permeable pouch format are typically used by placing one pouch containing the composition in the mouth of a human subject/user. Generally, the pouch is placed somewhere in the oral cavity of the user, for example under the lips, in the same way as moist snuff products are generally used. The pouch typically is not chewed or swallowed unless the pouch composition or materials are ingestible (e.g., dissolvable or dispersible) as described herein below. Exposure to saliva then causes some of the components of the composition therein (e.g., flavoring agents and/or nicotine) to pass through e.g., the water-permeable pouch and provide the user with flavor and satisfaction, and the user is not required to spit out any portion of the mixture. After about 10 minutes to about 60 minutes, typically about 15 minutes to about 45-minutes, of use/enjoyment, substantial amounts of the mixture have been ingested by the human subject, and the pouch may be removed from the mouth of the human subject for disposal.

Accordingly, in certain embodiments, the composition as disclosed herein and any other components noted above are combined within a moisture-permeable packet or pouch that acts as a container for use of the composition to provide a pouched product configured for oral use. Certain embodiments of the disclosure will be described with reference to the accompanying drawing, and these described embodiments involve snus-type products having an outer pouch and containing a mixture as described herein. As explained in greater detail below, such embodiments are provided by way of example only, and the pouched products of the present disclosure can include the composition in other forms. The mixture/construction of such packets or pouches, such as the container pouch 102 in the embodiment illustrated in the drawing may be varied. Referring to the drawing, there is shown a first embodiment of a pouched product 100. The pouched product 100 includes a moisture-permeable container in the form of a pouch 102, which contains a material 104 comprising a composition as described herein.

Suitable packets, pouches or containers of the type used for the manufacture of smokeless tobacco products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The mixture may be contained in pouches and packaged, in a manner and using the types of components used for the manufacture of conventional snus types of products. The pouch provides a liquid-permeable container of a type that may be considered to be similar in character to the mesh-like type of material that is used for the construction of a tea bag. Components of the mixture readily diffuse through the pouch and into the mouth of the user.

Non-limiting examples of suitable types of pouches are set forth in, for example, U.S. Pat. No. 5,167,244 to Kjerstad and 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which are incorporated herein by reference. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and mixture each may undergo complete dispersion within the mouth of the user during normal conditions of use, and hence the pouch and mixture both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Example pouch materials, though water dispersible or dissolvable, may be designed and manufactured such that under conditions of normal use, a significant amount of the mixture contents permeate through the pouch material prior to the time that the pouch undergoes loss of its physical integrity. If desired, flavoring ingredients, disintegration aids, and other desired components, may be incorporated within, or applied to, the pouch material.

The amount of material contained within each product unit, for example, a pouch, may vary. In some embodiments, the weight of the composition within each pouch is at least about 50 mg, for example, from about 50 mg to about 1 gram, from about 100 to 800 about mg, or from about 200 to about 700 mg. In some smaller embodiments, the weight of the composition within each pouch may be from about 100 to about 300 mg. For a larger embodiment, the weight of the composition within each pouch may be from about 300 mg to about 700 mg. If desired, other components can be contained within each pouch. For example, at least one flavored strip, piece or sheet of flavored water dispersible or water-soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and 6,923,981 to Leung et al.; and The EFSA Journal (2004) 85, 1-32; which are incorporated herein by reference.

A pouched product as described herein can be packaged within any suitable inner packaging material and/or outer container. See also, for example, the various types of containers for smokeless types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., U.S. Pat. No. D592,956 to Thiellier; U.S. Pat. No. D594,154 to Patel et al.; and U.S. Pat. No. D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference.

The types and amounts of additional ingredients contained within the oral compositions and products described herein will vary depending, e.g., upon the oral composition/product form and the desired attributes of the oral composition or product. Certain non-limiting optional additional components that can, in some embodiments, be incorporated within the disclosed oral compositions and products in addition to the disclosed resinate complex include, e.g., one or more further active ingredients, carriers/fillers, flavorants, taste modifiers, salts, sweeteners, binding agents, humectants, buffering agents, colorants, thickening agents, and various combinations thereof, as will be described herein below.

### Further Active Ingredients

In some embodiments, the oral composition and/or oral product provided according to the present disclosure comprises one or more further active ingredients/agents in addition to the substituted 3-(1-methylpyrrolidin-2-yl)pyridine, optionally substituted 3-(azetidin-2-yl)pyridine, optionally substituted 3-(azetidin-2-ylmethoxy)pyridine, or other active ingredient associated with the disclosed resinate complexes. The additional active agent may be a physiologically active material, which is a material intended to achieve or enhance a physiological response. The active agent may for example be selected from nutraceuticals, nootropics, psychoactives. The active agent may be naturally occurring or synthetically obtained. The active agent may comprise for example nicotine, caffeine, taurine, theanine, vitamins such as B6 or B12 or C, melatonin, cannabinoids, or constituents, derivatives, or combinations thereof. The active agent may comprise one or more constituents, derivatives or extracts of Cannabis or another botanical (other than tobacco).

### Nicotine

In some embodiments, the oral composition or product further comprises nicotine as an active agent. In some embodiments, this nicotine may be derived from a botanical extract included in the oral composition or product. In other embodiments, the nicotine is an additional active substance. In some embodiments, disclosed oral composition or product has a nicotine content of from about 1.5 wt % to about 7 wt % of the oral composition or product.

In some embodiments, the oral composition or product may comprise from at least about 1.5 wt %, about 2 wt %, about 2.5 wt %, about 3 wt %, about 3.5 wt %, about 4 wt %, about 4.5 wt % or about 5 wt % of nicotine on a dry weight basis. The oral composition or product may comprise no more than about 7 wt %, about 6.5 wt %, about 6 wt %, about 5.5 wt %, about 5 wt %, about 4.5 wt %, about 4 wt %, about 3.5 wt % or about 3 wt % of nicotine on a dry weight basis. For example, the oral composition or product associated with the disclosed oral composition or product may comprise from about 2 to about 6 wt %, or from about 4 to about 5 wt % nicotine by weight on a dry weight basis.

In some embodiments, the oral composition or product comprises from about 1 wt %, about 1.5 wt % or about 2 wt % to about 6 wt %, about 5 wt %, about 4 wt % or about 3 wt % of nicotine on a dry weight basis.

In some embodiments, the oral composition or product of the disclosure as a whole can be completely free or substantially free of nicotine (3-(1-methylpyrrolidin-2-yl)pyridine). By “substantially free” it is meant that no nicotine has been intentionally added, beyond trace amounts that may be present e.g., as an impurity in another component, including as a minor impurity in the substituted 3-(1-methylpyrrolidin-2-yl)pyridine. Cannabinoid

In some embodiments, the optional further active agent comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as Cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in Cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example Δ8- and Δ9-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is Δ8-CBD. In some embodiments, the CBD is Δ9-CBD.

In some embodiments, a cannabinoid (e.g., CBD) is incorporated within the disclosed oral composition or product in the form of an isolate. An isolate is an extract from a plant, such as Cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the oral composition or product is no greater than about 1% by weight on a dry weight basis, such as no greater than about 0.5% by weight, no greater than about 0.1% by weight, or no greater than about 0.01% by weight, based on the weight of the oral composition or product on a dry weight basis.

The choice of cannabinoid and the particular percentages thereof which may be present within the oral composition or product will vary depending upon the desired characteristics of the material.

In some embodiments, the cannabinoid (such as CBD) is present in the oral composition or product in a concentration of at least about 0.001% by weight of the oral composition or product, such as in a range from about 0.001% to about 2% by weight of the oral composition or product. In some embodiments, the cannabinoid (such as CBD) is present in the disclosed oral composition or product in a concentration of from about 0.1% to about 1.5% by weight, based on the total weight of the oral composition or product. In some embodiments, the cannabinoid (such as CBD) is present in a concentration from about 0.4% to about 1.5% by weight, based on the total weight of the oral composition or product.

Alternatively, or in addition to a cannabinoid, the active agent may include a cannabimimetic, which is a class of compounds derived from plants other than Cannabis that have biological effects on the endocannabinoid system similar to cannabinoids. Examples include yangonin, alpha-amyrin or beta-amyrin (also classified as terpenes), cyanidin, curcumin (turmeric), catechin, quercetin, salvinorin A, N-acylethanolamines, and N-alkylamide lipids. Such compounds can be used in the same amounts and ratios noted herein for cannabinoids.

### Terpenes

Further active agents suitable for use in certain disclosed oral compositions or products provided herein can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active agent comprises more than one terpene. For example, the active agent may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active agent and/or as a flavorant. The amount of terpene and/or cannabinoid present may vary accordingly based on their intended purpose.

In some embodiments, the optional further active agent comprises caffeine, melatonin, an amino acid, a vitamin, or combinations thereof. In some embodiments, the active agent comprises taurine, theanine, vitamin B6, B12, or C, melatonin, cannabinoids, or constituents, derivatives, or combinations thereof.

### Botanical

In some embodiments, the optional further active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the materials and/or articles as disclosed herein can be characterized as free of any tobacco material (e.g., any embodiment as disclosed herein may be completely or substantially free of any tobacco material). By “substantially free” is meant that no tobacco material has been intentionally added. For example, some embodiments can be characterized as having less than 0.001% by weight of tobacco, or less than 0.0001%, or even 0% by weight of tobacco (exclusive of any nicotine content, if present).

In some embodiments, the oral composition or product comprises a botanical in an amount from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the weight of the oral composition or product on a dry weight basis.

In some embodiments, the oral composition or product comprises one or more botanical materials in the form of one or more botanical extracts. In some embodiments, the botanical extract is a liquid or solid that has been isolated from a botanical material. The botanical materials used in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein.

Non-limiting examples of botanicals or botanical-derived materials include acai berry (Euterpe oleracea martius), acerola (Malpighia glabra), alfalfa, allspice, Angelica root, anise (e.g., star anise), annatto seed, apple (Malus domestica), apricot oil, ashwagandha, Bacopa monniera, baobab, basil (Ocimum basilicum), bay, bee balm, beet root, bergamot, blackberry (Morus nigra), black cohosh, black pepper, black tea, blueberries, boldo (Peumus boldus), borage, bugleweed, cacao, calamus root, camu (Myrcaria dubia), Cannabis/hemp, caraway seed, cardamom, cassis, catnip, catuaba, cayenne pepper, Centella asiatica, chaga mushroom, Chai-hu, chamomile, cherry, chervil, chive, chlorophyll, chocolate, cilantro, cinnamon (Cinnamomum cassia), citron grass (Cymbopogon citratus), citrus, clary sage, cloves, coconut (Cocos nucifera), coffee, comfrey leaf and root, cordyceps, coriander seed, cranberry, cumin, curcumin, damiana, dandelion, Dorstenia arifolia, Dorstenia odorata, Echinacea, elderberry, elderflower, endro (Anethum graveolens), evening primrose, eucalyptus, fennel, feverfew, flax, Galphimia glauca, garlic, ginger (Zingiber officinale), gingko biloba, ginseng, goji berries, goldenseal, grape seed, grapefruit, grapefruit rose (Citrus paradisi), graviola (Annona muricata), green tea, guarana, gutu kola, hawthorn, hazel, hemp, hibiscus flower (Hibiscus sabdariffa), honeybush, hops, jiaogulan, jambu (Spilanthes oleraceae), jasmine (Jasminum officinale), juniper berry (Juniperus communis), Kaempferia parviflora (Thai ginseng), kava, laurel, lavender, lemon (Citrus limon), lemon balm, lemongrass, licorice, lilac, Lion's mane, lutein, maca (Lepidium meyenii), mace, marjoram, matcha, milk thistle, mints (menthe), mulberry, Nardostachys chinensis, nutmeg, olive, oolong tea, orange (Citrus sinensis), oregano, papaya, paprika, pennyroyal, peppermint (Mentha piperita), pimento, potato peel, primrose, quercetin, quince, red clover, resveratrol, Rhizoma gastrodiae, Rhodiola, rooibos (red or green), rosehip (Rosa canina), rosemary, saffron, sage, Saint John's Wort, sandalwood, salvia (Salvia officinalis), savory, saw palmetto, Sceletium tortuosum, Schisandra, Silybum marianum, Skullcap, spearmint, Spikenard, spirulina, slippery elm bark, sorghum bran hi-tannin, sorghum grain hi-tannin, spearmint (Mentha spicata), spirulina, star anise, sumac bran, tarragon, thyme, tisanes, turmeric, Turnera aphrodisiaca, Uva ursi, valerian, vanilla, Viola odorata, white mulberry, wild yam root, wintergreen, Withania somnifera, yacon root, yellow dock, yerba mate, and yerba santa.

In some embodiments, the optional further active agent comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the weight of the oral composition or product on a dry weight basis.

In some embodiments, the optional further active agent comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In some embodiments, the active ingredient comprises Korean ginseng. In some embodiments, ginseng is present in an amount of from about 0.4 to about 0.6% by weight, based on the weight of the oral composition or product on a dry weight basis.

In some embodiments, the botanical extract is selected from eucalyptus, star anise, cocoa and hemp. In some embodiments, the botanical extract is selected from rooibos and fennel.

A botanical extract may be prepared by processing techniques such as expression (such as juicing or pressing) or solvent extraction. Optionally, the extract is concentrated and/or purified, for example by distillation. In some embodiments, the botanical material is macerated, frequently without heating, to soften and degrade the material prior to extraction. In some embodiments, the botanical extract is an aqueous extract, obtained by extraction with water. Additionally or alternatively, other solvents may be used, including supercritical fluids.

In some embodiments, the botanical extract is a tobacco extract. In some embodiments, the tobacco extract may be an aqueous extract, obtained by extraction with water. The tobacco extract may be an extract from any suitable tobacco, such as single grades or blends, cut rag or whole leaf, including Virginia and/or Burley and/or Oriental. It may also be an extract from tobacco particle ‘fines’ or dust, expanded tobacco, stems, expanded stems, and other processed stem materials, such as cut rolled stems. The extract may be obtained from a ground tobacco or a reconstituted tobacco material.

### Stimulant

In some embodiments, the optional further active agent comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active agent comprises caffeine. In some embodiments, the caffeine is present in an encapsulated form. On example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the weight of the oral composition or product on a dry weight basis. In some embodiments, the disclosed oral composition or product comprises caffeine in an amount of from about 1.5 to about 6% by weight, based on the weight of the oral composition or product on a dry weight basis.

### Amino Acids

In some embodiments, the optional further active agent comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamnylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the weight of the oral composition or product on a dry weight basis.

Vitamin and/or Mineral

In some embodiments, the optional further active agent comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine. In some embodiments, the active ingredient comprises one or more of vitamin B6 and B12. In some embodiments, the active ingredient comprises theanine and one or more of vitamin B6 and B12.

In some embodiments, the optional further active agent comprises vitamin A. In some embodiments, the vitamin A is encapsulated. In some embodiments, the active agent comprises vitamin B6, vitamin B12, vitamin E, vitamin C, or a combination thereof.

In some embodiments, the optional further active agent comprises a mineral. As used herein, the term “mineral” refers to an inorganic molecule (or related set of molecules) that is an essential micronutrient needed for the proper functioning of various systems in a mammal. Non-limiting examples of minerals include iron, zinc, copper, selenium, chromium, cobalt, manganese, calcium, phosphorus, sulfur, magnesium, and the like. In some embodiments, the active ingredient comprises iron. Suitable sources of iron include, but are not limited to, ferrous salts such as ferrous sulfate and ferrous gluconate. In some embodiments, the iron is encapsulated.

When present, a vitamin or mineral (or combinations thereof such as vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the weight of the oral composition or product on a dry weight basis.

### Antioxidant

In some embodiments, the optional further active agent comprises one or more antioxidants. As used herein, the term “antioxidant” refers to a substance which prevents or suppresses oxidation by terminating free radical reactions and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, Withania somnifera, Lion's mane, and Silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the weight of the oral composition or product on a dry weight basis.

### Pharmaceutical Ingredient

In some embodiments, the optional further active agent comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the weight of the oral composition or product on a dry weight basis.

In some embodiments, the oral composition or product is substantially free of any API. By “substantially free of any API” means that the material or article does not contain, and specifically excludes, the presence of any API as defined herein, such as any Food and Drug Administration (FDA) approved therapeutic agent intended to treat any medical condition.

### Carrier/Filler

Compositions and products as described herein can include at least one component that may be characterized as being a carrier component and/or a filler component. In some embodiments, the compositions may include both of a carrier and a filler, and various materials may fulfill the function of both a carrier and a filler. A filler component may fulfill multiple functions, such as enhancing certain organoleptic properties such as texture and mouthfeel, enhancing cohesiveness or compressibility of the product, and the like. Generally, filler components are porous and/or particulate materials. In some embodiments, the present compositions may comprise a carrier. In further embodiments, the present compositions may comprise a carrier and a filler. The carrier/filler may be configured in one or more embodiments to absorb and/or adsorb at least a portion of at least one component of the compositions and products.

In some embodiments, a carrier component and/or a filler component may be cellulose-based. For example, suitable particulate components are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential particulate carrier and/or filler components include maltodextrin, dextrose, calcium carbonate, calcium phosphate, lactose, mannitol, xylitol, and sorbitol. Combinations of materials can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “modified” starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate.

In some embodiments, a carrier component and/or a filler component may be a cellulose material or cellulose derivative. One particularly suitable material for use in the products described herein is microcrystalline cellulose (“mcc”). The mcc may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In one embodiment, a composition as described herein may comprise mcc as a particulate filler component and/or as a carrier component. The quantity of mcc present in the compositions as described herein may vary according to the desired properties. In some embodiments, a cellulose derivative or a combination of such derivatives in particular may be used in combination with a different carrier component, and this particularly can include cellulose derivatives, such as a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC.

In some embodiments, compositions and products as described particularly can include one or more materials that can be particularly useful as a carrier. In some embodiments, the carrier material can be particularly adapted to or configured to contribute to a controlled release of at least a portion of one or more components of the composition when the composition or product is present in the oral cavity of a consumer. In some embodiments, materials suitable for use as a carrier can include gelling agents and other materials that may otherwise be useful as a binder. For example, in some embodiments, pectins, gums, and alginates may be utilized as carrier materials that can be useful for carrying one or more components and providing for controllable release from the compositions and products as described herein.

The total amount of carrier component(s) and filler component(s) present in the composition can vary, but in some embodiments is typically up to about 75 percent of the composition by weight, based on the total weight of the composition. A typical range of total carrier and/or filler component within the composition can be from about 10 to about 75 percent by total weight of the composition, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the total amount of carrier/filler component is at least about 10 percent by weight, such as at least about 20 percent, or at least about 25 percent, or at least about 30 percent, or at least about 35 percent, or at least about 40 percent, based on the total weight of the composition.

### Flavoring Agents

In some embodiments, the oral composition or product as described herein comprises a flavoring agent. As used herein, a “flavoring agent” or “flavorant” is any flavorful or aromatic substance capable of altering the sensory characteristics associated with the disclosed oral composition or product. Examples of sensory characteristics that can be modified by the flavoring agent include taste, mouthfeel, moistness, coolness/heat, and/or fragrance/aroma. Flavoring agents may be imitation, natural, or synthetic or blends thereof. The character of the flavors imparted thereby may be described, without limitation, as fresh, sweet, herbal, confectionary, floral, fruity, or spicy.

Flavoring agents may include naturally occurring flavor materials, botanicals, extracts of botanicals, synthetically obtained materials, or combinations thereof, e.g., tobacco, Cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Specific types of flavors include, but are not limited to, vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, pineapple, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite and may be employed as concentrates or flavor packages (e.g., spearmint and menthol, orange and cinnamon, lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent generally comprises at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the oral composition or product comprises a sensate which provides to the user of such article a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA 4693), WS-116 (FEMA 4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N-(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entireties by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which is incorporated in its entirety by reference hereto. In some embodiments, the cooling agent comprises menthol, eucalyptus, mint, menthol, menthyl esters, eucolyptol, WS-3, WS-23, WS-5, (1R,2S,5R)—N-(4-(cyanomethyl)phenyl)menthylcarboxamide (Evercool™ 180), (1R,2S,5R)—N-(2-(pyridin-2-yl)ethyl)menthylcarboxamide (Evercool™ 190), or a combination thereof.

In some embodiments, the oral composition or product does not comprise a flavoring agent, and comprises only a cooling agent to provide the desired user experience. In some embodiments, the cooling agent is WS-3.

In some embodiments, oral composition or product comprises a modulator or sensate which provides to the user of such composition a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form.

The amount of flavoring agent utilized in the disclosed oral composition or product can vary, but is typically up to about 60% by weight based on the weight of the oral composition or product on a dry weight basis. For example, the material and/or article may comprise up to about 60 wt %, about 50 wt %, about 40 wt %, about 30 wt %, about 20 wt %, about 10 wt % or about 5 wt % of a flavoring agent based on the weight of the oral composition or product on a dry weight basis. In some embodiments, the material and/or article may comprise at least about 0.5 wt %, about 1 wt %, about 2 wt %, about 5 wt %, about 10 wt %, about 20 wt % or about 30 wt % of flavoring agent (based on the weight of the oral composition or product on a dry weight basis). For example, the material and/or article may comprise from about 10 to about 60 wt %, from about 20 to about 50 wt % or from about 30 to about 40 wt % of flavoring agent based on the weight of the oral composition or product on a dry weight basis.

In some embodiments, the flavor comprises menthol, spearmint and/or peppermint.

In some embodiments, the flavor comprises flavor components of cucumber, blueberry, citrus fruits and/or redberry.

In some embodiments, the flavor comprises eugenol.

In some embodiments, the flavoring agent (if present) comprises, consists essentially of, or consists of, menthol. In some embodiments, the oral composition or product does not comprise an added flavor.

### Taste Modifier

In order to improve the organoleptic properties of an oral composition or product as disclosed herein, the oral composition or product may include one or more taste modifying agents (“taste modifiers”) which may serve to mask, alter, block, or improve the flavor of a material as described herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the material and/or article comprises an active ingredient having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., an active ingredient). In some embodiments, the taste modifier is capsaicin.

In some embodiments, the taste modifier is the amino acid gamma-amino butyric acid (GABA), referenced herein above with respect to amino acids. Studies in mice suggest that GABA may serve function(s) in taste buds in addition to synaptic inhibition. See, e.g., Dvoryanchikov et al., J Neurosci. 2011 Apr. 13; 31(15):5782-91. Without wishing to be bound by theory, GABA may suppress the perception of certain tastes, such as bitterness. In some embodiments, the composition comprises caffeine and GABA.

In some embodiments, the taste modifier is adenosine monophosphate (AMP). AMP is a naturally occurring nucleotide substance which can block bitter food flavors or enhance sweetness. It does not directly alter the bitter flavor but may alter human perception of “bitter” by blocking the associated receptor.

In some embodiments, the taste modifier is lactisole. Lactisole is an antagonist of sweet taste receptors. Temporarily blocking sweetness receptors may accentuate e.g., savory notes.

When present, a representative amount of taste modifier is about 0.01% by weight or more, about 0.1% by weight or more, or about 1.0% by weight or more, but will typically make up less than about 10% by weight of the total weight of the oral composition or product on a dry weight basis (e.g., from about 0.01%, about 0.05%, about 0.1%, or about 0.5%, to about 1%, about 5%, or about 10% by weight of the weight of the oral composition or product on a dry weight basis).

### Salt

In some embodiments, the oral composition or product may further comprise a salt (e.g., an alkali metal salt, typically employed in an amount sufficient to provide desired sensory attributes to the material or article. Non-limiting examples of suitable salts include sodium chloride, potassium chloride, ammonium chloride, flour salt, and the like.

When present, a representative amount of salt is about 0.5 percent by weight or more, about 1.0 percent by weight or more, or at about 1.5 percent by weight or more, but will typically make up about 20 percent or less of the total weight of the composition, or about 15 percent or less or about 10 percent or less (e.g., about 0.5 to about 10 percent by weight) based on the weight of the oral composition or product on a dry weight basis.

### Sweetener

In order to improve the sensory properties of the oral composition or product according to the disclosure, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is sucralose, acesulfame K, or a combination thereof.

When present, a sweetener or combination of sweeteners may make up from about 0.01 to about 20% or more of the weight of the oral composition or product on a dry weight basis, for example, from about 0.01 to about 0.1, from about 0.1 to about 1%, from about 1 to about 5%, from about 5 to about 10%, or from about 10 to about 20% by weight, based on the weight of the oral composition or product on a dry weight basis. In some embodiments, a combination of sweeteners is present at a concentration of from about 0.01% to about 0.1%, such as about 0.01, about 0.02, about 0.03, about 0.04, about 0.05, about 0.06, about 0.07, about 0.08, about 0.09, or about 0.1% based on the weight of the oral composition or product on a dry weight basis. In some embodiments, a combination of sweeteners is present at a concentration of from about 0.1% to about 0.5% by weight of the composition, such as about 0.1, about 0.2, about 0.3, about 0.4, or about 0.5% based on the weight of the oral composition or product on a dry weight basis. In some embodiments, a combination of sweeteners is present at a concentration of from about 1% to about 3% based on the weight of the oral composition or product on a dry weight basis.

### Binding Agent

A binder (or combination of binders) can optionally be employed in the oral composition or product described herein in an amount sufficient to provide the desired physical attributes and physical integrity. The binder material can serve to add cohesiveness to a material and can also serve as gelling agents. Typically, the amount of binder present is up to about 50% by weight, and some embodiments are characterized by a binder content of at least about 5% by weight, based on the total weight of the oral composition or product on a dry weight basis. In some embodiments, the binder is present in an amount by weight in a range from about 5 to about 50% based on the total weight of the composition, such as from about 5%, about 10%, about 15%, about 20%, about 25%, or about 30%, to about 35%, about 40%, or about 45% by weight, based on the weight of the oral composition or product on a dry weight basis.

Typical binders can be organic or inorganic, or a combination thereof. Representative binders include povidone, sodium alginate, pectin, gums, carrageenan, pullulan, zein, cellulose derivatives, and the like, and combinations thereof. In some implementations, combinations or blends of two or more binder materials may be employed. Other examples of binder materials are described, for example, in U.S. Pat. No. 5,101,839 to Jakob et al.; and U.S. Pat. No. 4,924,887 to Raker et al., each of which is incorporated herein by reference in its entirety.

In some embodiments, the binder is selected from the group consisting of agar, alginates, carrageenan and other seaweed hydrocolloids, exudate gum hydrocolloids, cellulose ethers, starches, gums, dextrans, povidone, pullulan, zein, or combinations thereof.

In some embodiments, the binder is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). Suitable cellulose ethers include hydroxypropylcellulose, such as Klucel H from Aqualon Co.; hydroxypropylmethylcellulose, such as Methocel K4MS from DuPont; hydroxyethylcellulose, such as Natrosol 250 MRCS from Aqualon Co.; methylcellulose, such as Methocel A4M, K4M, and E15 from DuPont.; and sodium carboxymethylcellulose, such as CMC 7HF, CMC 7LF, and CMC 7H4F from Aqualon Co. In some embodiments, the binder is one or more cellulose ethers (e.g., a single cellulose ether or a combination of several cellulose ethers, such as two or three, for example). In some embodiments, the binder is a cellulose ether selected from the group consisting of methylcellulose, hydroxypropylcellulose, hydroxypropylmethylcellulose, hydroxyethyl cellulose, carboxymethylcellulose, and combinations thereof.

### Humectant

In some embodiments, one or more humectants may be employed in the oral composition or product described herein. Examples of humectants include, but are not limited to, polyols such as glycerin, propylene glycol, and the like. Where included, the humectant is typically provided in an amount sufficient to provide desired moisture attributes to the composition. When present, a humectant will typically make up about 20% or less of the weight of the oral composition or product on a dry weight basis or 15% or less of the weight of the oral composition or product on a dry weight basis (e.g., from about 1% to about 20% by weight or about 5% to about 15% by weight).

### Buffering Agent

In some embodiments, the oral composition or product of the present disclosure can comprise one or more pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Non-limiting examples of suitable buffers include alkali metals acetates, glycinates, phosphates, glycerophosphates, citrates, carbonates, hydrogen carbonates, borates, or mixtures thereof.

Where present, the buffering agent is typically present in an amount less than about 5 percent based on the weight of the oral composition or product on a dry weight basis, for example, from about 0.5% to about 20%, such as, e.g., from about 0.75% to about 15%, from about 1% to about 10%, or from about 1% to about 5% by weight, based on the weight of the oral composition or product on a dry weight basis.

### Colorant

A colorant may optionally be employed in amounts sufficient to provide the desired physical attributes to the oral composition or product. Examples of colorants include various dyes and pigments, such as caramel coloring and titanium dioxide. Natural colorants such as curcumin, beet juice extract, spirulina may be used; also, a variety of synthetic pigments may be used. The amount of colorant utilized in the material can vary, but when present is typically up to about 3% by weight, such as from about 0.1%, about 0.5%, or about 1%, to about 3% by weight, based on the weight of the oral composition or product on a dry weight basis.

### Oral Care Additive

In some embodiments, the material or article comprises an oral care ingredient (or mixture of such ingredients). Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the composition. Other examples of ingredients that can be incorporated in desired effective amounts within the present composition can include those that are incorporated within the types of oral care compositions set forth in Takahashi et al., Oral Microbiology and Immunology, 19(1), 61-64 (2004); U.S. Pat. No. 6,083,527 to Thistle; and US Pat. Appl. Pub. Nos. 2006/0210488 to Jakubowski and 2006/02228308 to Cummins et al. Other exemplary ingredients include those contained in formulations marketed as MALTISORB® by Roquette and DENTIZYME® by NatraRx. When present, a representative amount of oral care additive is at least about 1%, often at least about 3%, and frequently at least about 5% of the total dry weight of the oral composition or product on a dry weight basis. The amount of oral care additive within the material or article will not typically exceed about 30%, often will not exceed about 25%, and frequently will not exceed about 20% of the weight of the oral composition or product on a dry weight basis.

### Thickening Agent

In some embodiments, the oral composition or product will include one or more thickening agents to adjust the viscosity of the oral composition. Example thickening agents povidone, alginates, starches, pectin, carrageenan, pullulan, zein, natural gums, cellulose derivatives and the like, and combinations thereof. In some embodiments, the thickening agent is a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. In some embodiments, the thickening agent comprises a cellulose derivative or a combination of such derivatives. Example cellulose derivatives include cellulose ethers (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”).

A thickening agent may be employed in an amount sufficient to provide the desired physical attributes, such as viscosity, to the composition. The amount of thickening agent utilized in the composition can vary, but is typically up to about 10 weight percent, and certain embodiments are characterized by a thickening agent content of at least about 0.1% by weight, such as about 1 to about 10% by weight, or about 2 to about 5% by weight, based on the total weight of the oral composition or product.

### Other Additives

Other additives can optionally be included in the disclosed materials and articles. The additives can be artificial or can be obtained or derived from herbal or biological sources. Examples of further types of additives include thickening or gelling agents (e.g., fish gelatin), emulsifiers, preservatives (e.g., potassium sorbate and the like), disintegration aids, or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference.

Typical inclusion ranges for such additional additives can vary depending on the nature and function of the additive and the intended effect on the final material or article, with an example range of up to about 10% by weight, based on the weight of the oral composition or product on a dry weight basis (e.g., about 0.1 to about 5% by weight).

The aforementioned additives can be employed together (e.g., as additive formulations) or separately (e.g., individual additive components can be added at different stages involved in the preparation of the treated material). Furthermore, the aforementioned types of additives may be encapsulated as provided in the final product or composition. Example encapsulated additives are described, for example, in WO2010/132444 to Atchley, which has been previously incorporated by reference herein.

Many modifications and other embodiments of the disclosure will come to mind to one skilled in the art to which this disclosure pertains having the benefit of the teachings presented in the foregoing description. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

### Preparation of Resinate Complex

About 4.39 g of a 12% aqueous 2-methyl-5-(1-methylpyrrolidin-2-yl)pyridine solution was added to about 6.14 g of powdered Amberlite® IRP64 resin (hydrogen form). The components were hand-mixed and the resulting mixture (“Mixture A”) was analyzed by extraction with 5% NaOH in water and quantification using UPLC with a PDA detector. The mixture was found to contain about 4.9% 2-methyl-5-(1-methylpyrrolidin-2-yl)pyridine.

### Preparation of Oral Composition

The oral composition was made from the composition below in Table 2. The pH of the oral composition (fill) was determined to be 6.87.

TABLE 2
Oral Composition

| Component | wt % |
|---|---|
| Microcrystalline Cellulose | 71.4 |
| Sodium Chloride | 3.9 |
| Water | 6.2 |
| Mixture A | 3.1 |
| Propylene Glycol | 8.8 |
| Aqueous Sodium Hydroxide (5M) | 1.8 |
| Xylitol | 2.9 |
| Sucralose | 0.5 |
| Sodium Benzoate | 0.4 |
| Flavor (Mint) | 0.9 |

### Preparation of Oral Product (Pouch)

A pouch was prepared by adding about 336 mg of the Oral Composition to a 30×12 cm cellulosic fleece pouch and adding about 68 mg of deionized water, giving a final pouch weight of 467 mg and 0.52 mg 2-methyl-5-(1-methylpyrrolidin-2-yl)pyridine. The pH of the resulting pouch was found to be 6.94.

What is claimed is:

1. A product for oral use comprising a resinate complex, the resinate complex comprising a resin base component and one or more active agents associated with the resin base component, selected from:
(i) a substituted 3-(1-methylpyrrolidin-2-yl)pyridine having a structure according to
Formula I: 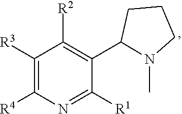
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen;
(ii) a substituted 3-(1-methylpyrrolidin-2-yl)pyridine having a structure according to Formula II: 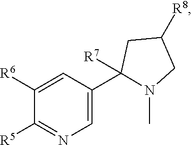
wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; and
(iii) a 3-(azetidin-2-yl)pyridine or 3-(azetidin-2-ylmethoxy)pyridine having a structure according to Formula III: 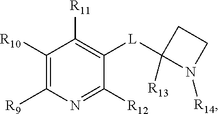
wherein L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The oral product of claim 1, wherein the active ingredient has a structure according to Formula I, optionally wherein R¹, R², and R³ are each H, and R⁴ is optionally substituted C₁-C₆ alkyl, F, Cl, Br, OCH₃, OEt, or CN, or optionally wherein R¹, R², and R³ are each H, and R⁴ is C₁-C₃ alkyl.

3. The oral product of claim 1, wherein the active ingredient has a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

4. The oral product of claim 1, wherein the active ingredient has a structure according to Formula III, optionally wherein R⁹ is H or CH₃; and R¹⁰, R¹¹, and R¹² are each H.

5. The oral product of claim 1, wherein the oral product further comprises a flavorant (e.g., a sensate), a salt, a colorant, a sweetener, a pH-adjusting component (buffering agent or pH adjuster), or any combination thereof.

6. The oral product of claim 1, wherein the oral product further comprises a filler, binder, disintegration aid, antioxidant, humectant, preservative, or any combination thereof.

7. The oral product of claim 1, wherein the oral product further comprises one or more additional active agents selected from the group consisting of a nicotine component, a cannabinoid, a terpene, caffeine, an amino acid, a vitamin, melatonin, a botanical extract, or any combination thereof.

8. The oral product of claim 1, wherein the oral product further comprises a nicotine component.

9. The oral product of claim 1, wherein the oral product is substantially free of a nicotine component.

10. The oral product of claim 1, in a form selected from the group consisting of a tablet, pastille, pouched product, gel, chewing gum, and meltable product.

11. The oral product of claim 10, in the form of a dissolvable tablet.

12. The oral product of claim 10, in the form of a pouched product, the pouched product comprising an outer, water-permeable pouch defining a cavity and an oral composition for oral use situated within the cavity, wherein the oral composition comprises the resinate complex.

13. The oral product of claim 12, wherein the oral composition further comprises one or more fillers in an amount of about 50% or more by weight.

14. The oral product of claim 12, wherein the oral composition further comprises a portion of the one or more active agents in released form, such that they are not associated with the resin base component.

15. The oral product of claim 14, wherein the one or more active agents in released form are in a form selected from free base form, salt form, co-crystal form, salt co-crystal form, ion-paired form, and combinations thereof.

16. A resinate complex, the resinate complex comprising a resin base component and one or more active agents associated with the resin base component, selected from:
(iv) a substituted 3-(1-methylpyrrolidin-2-yl)pyridine having a structure according to Formula I: 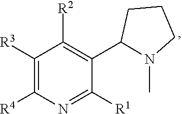
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen;
(v) a substituted 3-(1-methylpyrrolidin-2-yl)pyridine having a structure according to Formula II: 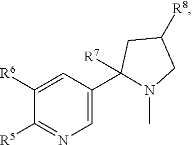
wherein R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; and
(vi) a 3-(azetidin-2-yl)pyridine or 3-(azetidin-2-ylmethoxy)pyridine having a structure according to Formula III: 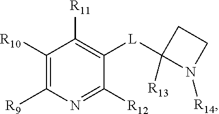
wherein L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

17. The resinate complex of claim 16, wherein the active ingredient has a structure according to Formula I, optionally wherein R¹, R², and R³ are each H, and R⁴ is optionally substituted C₁-C₆ alkyl, F, Cl, Br, OCH₃, OEt, or CN, or optionally wherein R¹, R², and R³ are each H, and R⁴ is C₁-C₃ alkyl.

18. The resinate complex of claim 16, wherein the active ingredient has a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

19. The resinate complex of claim 16, wherein the active ingredient has a structure according to Formula III, optionally wherein R⁹ is H or CH₃; and R¹⁰, R¹¹, and R¹² are each H.
